(19) United States
(12) Patent Application Publication
(10) Pub. No.: US 2013/0296182 A1
(43) Pub. Date: Nov. 7, 2013

(54) VARIABILITY SINGLE NUCLEOTIDE POLYMORPHISMS LINKING STOCHASTIC EPIGENETIC VARIATION AND COMMON DISEASE

(71) Applicants: Andrew P. Feinberg, Lutherville, MD (US); Jeffrey T. Leek, Annapolis, MD (US); Thor Aspelund, Reykjavik (IS); Vilmundur Gudnason, Kopavogur (IS); M. Daniele Fallin, Baltimore, MD (US); Rafael A. Irizarry, Baltimore, MD (US)

(72) Inventors: Andrew P. Feinberg, Lutherville, MD (US); Jeffrey T. Leek, Annapolis, MD (US); Thor Aspelund, Reykjavik (IS); Vilmundur Gudnason, Kopavogur (IS); M. Daniele Fallin, Baltimore, MD (US); Rafael A. Irizarry, Baltimore, MD (US)

(21) Appl. No.: 13/818,644

(22) PCT Filed: Aug. 31, 2011
(86) PCT No.: PCT/US11/50002

§ 371 Date: Jul. 23, 2013

Related U.S. Application Data
(60) Provisional application No. 61/382,826, filed on Sep. 14, 2010.
(60) Provisional application No. 61/378,762, filed on Aug. 31, 2010.

(51) Int. Cl. G06F 19/18; C12N 15/10; C12Q 1/68

(52) U.S. Cl. CPC G06F 19/18; C12Q 1/6883; C12N 15/1072
USPC 506/9; 435/6.11; 506/16; 702/19

(57) ABSTRACT

Provided are methods and models for an alternative source of disease risk, which identifies not genetic variants for a phenotype per se, but variants for variability itself. Also provided are methods and models for a genome-scale, gene-specific analysis of DNA methylation in the same individuals over time, in order to identify a personalized epigenomic signature that may correlate with common genetic disease. Also provided are methods and models for simulating stochastic epigenetic variation as a driving force of development, evolutionary adaptation, and disease.

### GRANT INFORMATION

This invention was made in part with government support under NIH Grant Nos. P50HG003233 and 2P50HG003323. The United States government has certain rights in this invention.

### BACKGROUND OF THE INVENTION

1. Field of the Invention

The invention relates generally to the field of epigenomics and more specifically to personal epigenomic analysis.

2. Background Information

First, the basis of modern disease association studies can be predicated on the “common disease common variant hypothesis,” which argues that frequent variants in the general population, that arose at a point of historical population restriction, are associated with genetic variants for common disease. The concept is rooted in the neo-Darwinian synthesis of the previous century, and the population genetic analysis of R. A. Fisher, who argued that complex (multigenic) phenotypes arise additively from individual quantitative trait loci (QTLs). A great deal of effort has been expended on finding associations of common disease with single nucleotide polymorphisms (SNPs). While there have been important successes, the overwhelming majority of GWAS studies have shown associations characterized by low odds ratios, around 70% report odd-ratio below 2, with generally relatively weak genome-wide statistical significance. This is a well-recognized problem in the GWAS community, and has led to discussions of sources of the missing “dark matter” of heritability, reviewed recently in the literature. Alternatives include copy number variants, and rare variants, although copy numbers also appear to account for a relatively small attributable risk of disease, e.g. <1% in schizophrenia. A major goal of funding agencies is to extend sequencing efforts to much larger cohorts, and the identification of the major cause of disease-related genetic variation is essential to fulfill ambitions for personalized medicine, i.e., targeting therapy and disease risk mitigation based on one's genome.

Second, a role for epigenetics in common disease has long been suspected, and a strong relationship with cancer has been shown. It is likely that common disease involves both genetic and epigenetic factors and that epigenetic modification could mark both environmental effects as well as mediate genetic effects. In addition to particular exposure-epigenetic relationships, epigenetic changes with aging support the notion that there is an environmental component to epigenetic variation. Studies of identical twins show greater differences in global DNA methylation in older than in younger twins, consistent with an age-dependent progression of epigenetic change. Global methylation changes over an 11 year span in participants of an Icelandic cohort, and age- and tissue-related alterations in some CpG islands from an array of 1,413 arbitrarily chosen CpG sites near gene promoters, further corroborate the evidence for dynamic methylation patterns over time. Other work, however, has suggested that epigenetic marks, or their maintenance, are themselves controlled by genes, and are thus heritable in the traditional sense and associated with particular DNA variants. This would predict that methylation marks are stable, rather than varying as controlled by changing environments.

Third, a key tenet of Origin of Species argues that phenotype is the result of many discrete traits that are individually and exquisitely selected, to quote Darwin, “detecting the smallest grain in the balance of fitness,” which has been described as Newtonian in its dependence on static forces acting in consistent ways. This concept is the basis for quantitative trait loci that has been proposed in the scientific field. This concept has led to the modern basis of population genetics that continuous variation exists within a population, yet selection is on individuals, which has led to models of balancing or purifying selection at the extremes of phenotype. The classic model also has significant limitations in explaining common human disease; common variants can explain only a small fraction of a given disease phenotype, even the most well understood, such as adult-onset diabetes and height.

Epigenetics, the study of nonsequence-based changes in DNA and associated proteins, was first suggested to play a role in evolution through Lamarckian inheritance, that is, direct modification of the genome by the environment, which is then transmitted transgenerationally. Two examples are commonly cited: changes in coat color caused by dietary modifications of DNA methylation of the agouti gene in mice and methylation of the axin-fused allele in kinked tail mice. Both of these examples involve methylation of a retrotransposon LTR sequence, and thus fit into various genetic exceptions to classical Darwinian thinking, including anticipation due to trinucleotide repeat expansion and lateral gene transfer in the evolution of influenza strains. But they have not been shown to be general mechanisms for either speciation or developmental differences across species, so-called “evo-devo,” or for canalization, a term coined to refer to a mechanism by which environmental perturbations during development are corrected by the genetic program, leading to a consistent developmental plan.

Indeed, canalization remains a “black box,” as noted by some in the scientific field. Others have discussed the potential role for Lamarckian inheritance in disease; for example, some have proposed a model of transgenerational epigenetic Lamarckian inheritance and noted that such modifications must persist for many generations to contribute substantially to average risk, which has implications for public health management. Although not disputing an important contribution of Lamarckian inheritance, here the invention provides an alternative view in which genetic modification could provide stochastic phenotypic variation favored by selection in changing environments, and also provide an alternative non-Lamarckian role for epigenetics in evolution.

Thus, there is a need for an alternative source of disease risk, which identifies not genetic variants for a phenotype per se, but variants for variability itself. There is also a need for a genome-scale, gene-specific analysis of DNA methylation in the same individuals over time, in order to identify a personalized epigenomic signature that may correlate with common genetic disease. There is also a need for a new model for simulating stochastic epigenetic variation as a driving force of development, evolutionary adaptation, and disease.

### SUMMARY OF INVENTION

First, the invention relates to variability single nucleotide polymorphisms (vSNPs) linking stochastic epigenetic variation and common disease. A major puzzle in human genetics is the relatively small attributable risk of common disease explained by common sequence variants, with most genome-wide association studies (GWAS) showing low odds ratios. The invention provides alternative models where genetic variants for stochastic epigenetic variation would confer an evolutionary selective advantage in changing environments, but could also increase disease risk in a given environment.

Accordingly, in one embodiment, the invention provides a method of predicting risk for a condition or disorder in a subject. The method includes: (a) measuring the expression level of at least one expression variable trait loci (eVTL) in a biological sample from the subject; (b) measuring the methylation level of at least one variably methylated region (VMR) correlated with at least one variability genotype in a biological sample from the subject; and (c) predicting the risk for the condition or disorder in the subject based on the expression level of the eVTL in (a) and the methylation level measured in (b).

In various embodiments, the method of the invention further includes performing an association study between a genotype variability information and a gene expression variability information, thereby identifying at least one variability genotype associated with the selected gene expression. In various embodiments, the method of the invention further includes the step of: performing an association study between each of the at least one variability genotype and a genome-wide gene expression data, thereby identifying at least one expression variable trait loci (eVTL), wherein the at least one eVTL is associated with the condition or disorder.

In another embodiment, the invention provides a method of predicting risk for a condition or disorder in a subject. The method includes: (a) obtaining genotype data from a plurality of samples; (b) obtaining genome-wide gene expression data from the samples; (c) performing a first variability test for the genotype data, thereby obtaining genotype variability information; (d) performing a second variability test for at least one selected gene expression from the samples, thereby obtaining gene expression variability information, wherein the selected gene expression correlates with the condition or disorder; (e) performing a first association study between the genotype variability information of (c) and the gene expression variability information of (d), thereby identifying at least one variability genotype associated with the selected gene expression; (f) performing a second association study between each of the at least one variability genotype identified in (e) and the genome-wide gene expression data of (b), thereby identifying at least one expression variable trait loci (eVTL), wherein the at least one eVTL is associated with the condition or disorder; (g) identifying a plurality of variably methylated regions (VMRs) correlated with the selected gene expression; (h) performing a linkage disequilibrium (LD) study between the at least one variability genotype identified in (e) and the VMRs correlated with the selected gene expression identified in (g), thereby identifying at least one VMR correlated with the variability genotype; (i) measuring expression level of the at least one eVTL in (f) in a biological sample from the subject; (j) measuring methylation level of the at least one VMR correlated with the variability genotype identified in (g) in a biological sample from the subject; and (k) predicting the risk for the condition or disorder in the subject based on the expression level of the eVTL in (i) and the methylation level measured in (j).

In various embodiments, the method further includes a step of performing a third association study between the genotype data of (a) and the selected gene expression from the samples, thereby identifying at least one mean genotype associated with the selected gene expression.

In another embodiment, the invention provides a method for analyzing epigenetic information, using suitable computer software for use on a computer. The method includes: (a) performing a first variability test for genotype data obtained from a plurality of samples, thereby obtaining genotype variability information; (b) performing a second variability test for at least one selected gene expression from the samples, thereby obtaining gene expression variability information; (c) performing a first association study between the genotype variability information of (a) and the gene expression variability information of (b), thereby identifying at least one variability genotype associated with the selected gene expression; (d) performing a second association study between each of the at least one variability genotype identified in (c) and genome-wide gene expression data obtained from the samples, thereby identifying at least one expression variable trait loci (eVTL); and (e) performing a linkage disequilibrium (LD) study between the at least one variability genotype identified in (c) and a plurality of variably methylated regions (VMRs) correlated with the selected gene expression, thereby identifying at least one VMR correlated with the variability genotype.

In various embodiments, the method of the invention further includes the step of performing a third association study between the genotype data and the selected gene expression from the samples, thereby identifying at least one mean genotype associated with the selected gene expression. In various embodiments, the method of the invention further includes performing a gene ontology analysis for each of the at least one variability genotype.

In another embodiment, the invention provides a system for identifying expression variable trait loci (eVTL) and variably methylated regions (VMRs) for predicting risk for a condition or disorder in a subject. The method includes: (a) a first variability module performing a first variability test for genotype data obtained from a plurality of samples, thereby obtaining genotype variability information; (b) a second variability module performing a second variability test for at least one selected gene expression, thereby obtaining gene expression variability information, wherein the selected gene expression correlates with the condition or disorder; (c) a first association module performing a first association study between the genotype variability information of (a) and the gene expression variability information of (b), thereby identifying at least one variability genotype associated with the selected gene expression; (d) a second association module performing a second association study between each of the at least one variability genotype identified in (c) and genome-wide gene expression data obtained from the samples, thereby identifying at least one expression variable trait loci (eVTL); and (e) a linkage disequilibrium module performing a linkage disequilibrium (LD) study between the at least one variability genotype identified in (c) and a plurality of VMRs correlated with the selected gene expression, thereby identifying at least one VMR correlated with the variability genotype.

In various embodiments, the system of the invention further includes a third association module performing a third association study between the genotype data and at least one selected gene expression from the samples, thereby identifying at least one mean genotype associated with the selected gene expression, wherein the selected gene expression correlates with the condition or disorder. In various embodiments, the system of the invention further includes a gene ontology module performing a gene ontology analysis for each of the at least one variability genotype.

Second, the invention also relates to personalized epigenomic signatures stable over time and covarying with body mass index. The present invention provides methods for predicting risk for a condition or disorder in a subject and methods for generating an epigenetic signature for a subject. The methods provided can be used to identify the risk of all the common diseases, and in particular instance, obesity. Also, the methods provided can be used to target the genes involved.

Accordingly, in one embodiment, the present invention provides a method for predicting risk for a condition or disorder in a subject over time. The method includes: (a) measuring intra-sample change over time for genome-wide variably methylated regions (VMRs) from a plurality of samples; (b) performing gene ontology analysis for the VMRs; (c) identifying at least one VMR correlated with the condition or disorder using a linear regression model; (d) measuring methylation level of the at least one VMRs correlated with the condition or disorder in a biological sample from the subject; and (e) predicting the risk for the condition or disorder in the subject based on the methylation level measured in (d).

In one embodiment, the present invention provides a method for generating an epigenetic signature for a subject. The method includes: (a) measuring intra-sample change over time for genome-wide variably methylated regions (VMRs) from a plurality of samples; (b) separating selected VMRs into two groups using a two component Gaussian mixture model based on the measured intra-sample change of (a), wherein the VMRs in the higher distribution are designated as dynamic VMRs and the VMRs in the lower distribution are designated as stable VMRs; (c) measuring methylation levels of a plurality of stable VMRs in a biological sample from the subject; and (d) generating the epigenetic signature for the subject based on the methylation levels measured in (c).

Third, the invention also relates to stochastic epigenetic variation as a driving force of development, evolutionary adaptation, and disease. Accordingly, the present invention provides a method for simulating epigenetic plasticity across generations. The method includes: (a) generating a plurality of genotype variants, wherein the genotype variants are genetically inherited; (b) applying natural selection favoring a first subset of the genotype variants; (c) enabling a plurality of stochastic epigenetic elements, wherein the stochastic epigenetic elements change phenotypes without changing the genotype variants; (d) allowing a changing environment across generations favoring a second subset of the genotype variants; and (e) monitoring fluctuations of mean phenotype across generations.

In various embodiments, the method of the invention further includes comparing frequency of fitness from genome-wide association study (GWAS) with the genotype variants which change the mean phenotype. In one embodiment, a Fisher-Wright neutral selection model is used. In another embodiment, a Fisher's additive model is used. In another embodiment, a multinomial distribution is used. In another embodiment, each of the genotype variants has two possible polymorphisms. In another embodiment, the stochastic epigenetic elements represent additions or deletions of CpG islands. In another embodiment, the method uses suitable computer software for use on a computer.

In another embodiment, the present invention provides a system for performing a method of the present invention. The system includes at least one computer readable medium having executable code with functionality for performing statistical algorithms and at least one database storing gene related or other biological information.

In another embodiment, the present invention provides a plurality of nucleic acid sequences, selected from the variably methylated region (VMR) sequences as set forth in Table 4, and any combination thereof. In one embodiment, the plurality is a microarray.

In another embodiment, the present invention provides a kit for detecting risk of a condition or disorder. The kit includes a plurality of oligonucleotide primer sequences capable of generating a plurality of amplificates from genomic DNA, the amplificates including variably methylated region (VMR) sequences as set forth in Table 4, and any combination thereof. The kit may further include instructions for detecting risk. In one embodiment, the condition or disorder is diabetes or obesity. In a related embodiment, the kit may further include computer executable code and instructions for performing statistical analysis.

### BRIEF DESCRIPTION OF THE DRAWINGS

For more complete understanding of the features and advantages of the present invention, reference is now made to the detailed description of the invention along with the accompanying figures, wherein:

FIG. 1 shows an exemplary flowchart for an embodiment of the invention.

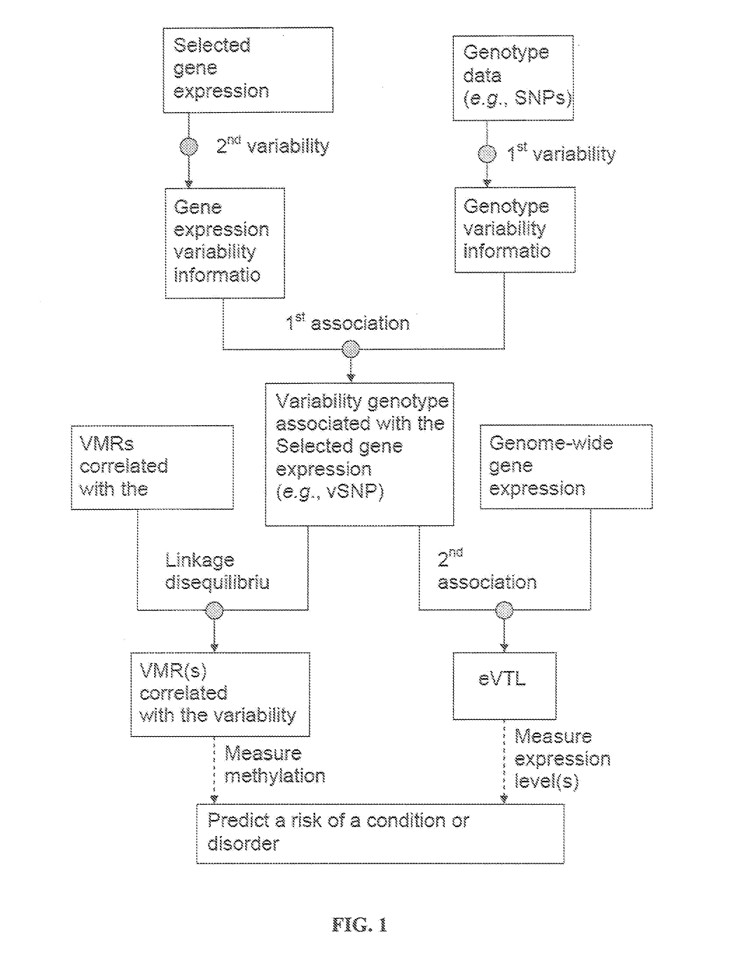

FIG. 2 is a series of graphical representations. FIG. 2A is a plot of m-SNP identified by analysis of the GoKinD dataset. FIG. 2B is a plot of significant variance SNP (vSNP) identified by analysis of the GoKinD dataset. FIG. 2C is a plot of the −log₁₀ p-values versus genomic position (chromosomes 1-22, X ordered from left to right) or mSNPs. FIG. 2D is a plot of the −log₁₀ p-values versus genomic position (chromosomes 1-22, X ordered from left to right) or vSNPs. FIG. 2E is a plot of the −log₁₀ p-values versus genomic position for expression variable trait loci (eVTL).

FIG. 3 is a pictorial representation of expression variable trait loci being located near variability methylated regions.

FIG. 4 is a series of graphical representations. The top panel depicts the distribution of HbA1c and the bottom panel depicts that relationship between HbA1c and methylation at VMRs in linkage disequilibrium for three HbA1c vSNPs near genes. FIG. 4A is of FGF3. FIG. 4B is of KCNQ1. FIG. 4C is of PER1.

FIG. 5 is a series of pictorial representations depicting the relationship between the new variability model and common disease. FIG. 5A is a series of illustrations of how mSNPs and vSNPs would affect disease status through a quantitative trait. FIG. 5B is an illustration of expected effect of mSNP and vSNP sizes detected by quantitative trait analysis, case-control analysis, and the variance procedure of the invention.

FIG. 6 is a graphical plot of the distribution of intra-individual change over time at VMRs.

FIG. 7 is a series of dendrograms. FIG. 7A is a dendrogram based on clustering applied to methylation profiles at all 227 VMRs. FIG. 7B is a dendrogram based on clustering applied to methylation profiles using only the 119 stable VMRs. Numbers represent individual IDs.

FIG. 8 is a series of methylation curves. Dashed lines are individual methylation curves. Solid lines are average curves by obese and normal groups. Bold straight lines, at the bottom of upper two boxes, indicate the boundaries of the VMR. CpG density is shown with CpG islands as a bold straight line at the bottom of the third box from the top. Gene location shown at bottom.

FIG. 9 is a series of graphical plots correlating methylation and BMI at six BMI-related VMRs. Points are individual IDs. Solid lines indicate visit 6 (first visit), and dotted lines indicate visit 7 (second visit).

FIG. 10 is a series of paired plots. In each paired plot, the top panel plots estimated methylation levels from various biological replicates from three different tissues: brain, liver, and spleen (dashed lines). The thicker solid lines represent the average curves for each tissue. The bars denote the regions in which the statistical method detected a VMR. The bottom panel highlights the liver. Only the four liver curves are shown. The different line types represent the four individual mice. FIG. 10A is of Bmp7. FIG. 10B is of Pou3f2. FIG. 10C is of Ntrk3. Each gene is involved in early embryogenic programming and bone induction, neurogenesis and stem cell reprogramming, and body position sensing, respectively.

FIG. 11 is a graphical plot depicting the association of VMRs with variability in gene expression of nearby genes. The human liver VMRs detected with the statistical algorithm of the invention are divided into three types: low variation (lowest 70%), high variation (highest 5%), and medium variation (the remainder). The VMRs within 500 bases from a gene's transcription start site are associated with that gene. The expression measurements are obtained for the same human livers, and the SD across subjects is used to quantify variability. These boxplots show the distribution of this variability stratified by VMR variability. The first boxplot represents genes not associated with a VMR.

FIG. 12 is a series of paired plots. Labeling is as in FIG. 10. FIG. 12A is of Bmpr2. FIG. 12B is of Irs1.

FIG. 13 is a series of paired plots. Labeling is as in FIG. 10. FIG. 13A is of Ptp4a1. FIG. 13B is of FOXD2.

FIG. 14 is a series of graphical representations. A 7,500-bp human region was mapped to the mouse genome. The x-axis shows an index so that mapped bases are on top of one another. Top Panel: Methylation profiles for each human sample. As in FIG. 10, the dashed lines represent the individuals, and the solid lines represent the tissue averages. Middle Panel: The same plot for mouse. Bottom Panel: Ticks representing CpG locations for human and mouse. The ticks represent CpGs that were conserved. The curves represent CpG counts in a moving window of size 200 bases. Shown is LHX1, a transcriptional regulator essential for vertebrate head organization and mesoderm organization.

FIG. 15 is a series of graphical representations. FIG. 15A plots simulations of natural selection. For each simulation, the population average and SD of the phenotype are computed as a function of generation. Two simulations are shown: simulation 1, natural selection in a fixed environment favoring positive Y but including a novel stochastic epigenetic element, such that eight mutations affect average Y and eight mutations affect variance of Y, and simulation 2, similar to simulation 1 but in this case allowing a changing environment across generations that favor at times positive Y and at times negative Y. The top panel shows the average (across all iterations) population average of Y as a function of generation for simulation 1 (solid lines) and simulation 2 (dot lines). The dashed vertical lines indicate the generations at which the environment is changed in simulation 2. The bottom panel shows the average (across all iterations) population standard deviation of Y. Note that with a changing environment, the average Y fluctuates around a common point, but the SD of Y increases consistently. FIG. 15B is a histogram depicting an emulation of GWAS analysis based on simulation 2 (varying variance of Y). Observed odds ratios are for SNPs that change the mean phenotype.

### DETAILED DESCRIPTION OF THE INVENTION

The invention relates to variability single nucleotide polymorphisms linking stochastic epigenetic variation and common disease. The present invention provides methods of predicting risk for a condition or disorder in a subject. Also provided are methods for analyzing epigenetic information, using suitable computer software for use on a computer. In addition, the present invention provides systems for identifying expression variable trait loci (eVTL) and variably methylated regions (VMRs) for predicting risk for a condition or disorder in a subject.

Further, the invention also relates to personalized epigenomic signatures. The present invention provides methods for predicting risk for a condition or disorder in a subject and methods for generating an epigenetic signature for a subject. The methods provided can be used to identify the risk of all the common diseases, and in a particular instance, obesity. Also methods provided can be used to target the genes involved. At least 14 genes have been identified in the present invention for particular diagnosis and also new target therapy to mitigate risk.

The invention also relates to stochastic epigenetic variation as a driving force of development, evolutionary adaptation, and disease. The present invention provides methods for simulating epigenetic plasticity across generations.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

In one embodiment, the invention relates to variability single nucleotide polymorphisms linking stochastic epigenetic variation and common disease. As such, in one embodiment, the invention relates to a method of predicting risk for a condition or disorder in a subject. The method includes (a) measuring the expression level of at least one expression variable trait loci (eVTL) in a biological sample from the subject; (b) measuring the methylation level of at least one variably methylated region (VMR) correlated with at least one variability genotype in a biological sample from the subject; and (c) predicting the risk for the condition or disorder in the subject based on the expression level of the eVTL in (a) and the methylation level measured in (b).

In one embodiment, the method of the invention further includes performing an association study between a genotype variability information and a gene expression variability information. In another embodiment, the method of the invention further includes the step of: performing an association study between each of the at least one variability genotype and a genome-wide gene expression data, thereby identifying at least one expression variable trait loci (eVTL), wherein the at least one eVTL is associated with the condition or disorder.

The alternative models of the invention were tested methods discussed in the Examples, identifying 282 variability-associated single-nucleotide-polymorphisms (vSNPs), at a false discovery rate threshold of 5%, affecting variance of hemoglobin A1C, a measure of chronic glucose levels; only 5 conventional mean phenotype SNPs (which the inventors term mSNPs are identified at the same FDR threshold in these data). The inventors confirmed the generality of vSNPs using gene expression data and genotypes from 210 HapMap individuals, with variability in gene expression itself as the phenotype. The inventors further found that vSNPs for gene expression, as well as known mSNPs found by common disease GWAS, are highly enriched (P=1.1×10⁻⁸ and P<1×10⁻¹⁶, respectively) in the vicinity of VMRs in the human genome. Further, in an independent sample of 65 individuals for whom genome-wide DNA methylation data had been measured, the inventors confirmed that the genotypes for 3 of the identified vSNPs are associated with differences in variability of HbA1c, which is also correlated with DNA methylation. The invention provides that some of the “dark matter” of variability in phenotype is hidden in plain view and will be accessible by complementary epigenetic analysis.

Disease variants are usually identified by searching for single nucleotide polymorphisms (SNPs) that are associated with differences in the average disease phenotype. The invention provides alternative models that SNPs may be associated with changes in variability of phenotype, which are designated as vSNPs. The invention provides a new evolutionary model that is based on inherited epigenetic variability.

While the methods of the invention have been exemplified by investigating diabetes and obesity, any number of disorders may be investigated and identified using the methods described herein. As used herein, the term “disorder” or “disease” is used to refer to a variety of pathologies. For example, the term may include, but is not limited to, various metabolic disorders of carbohydrate, lipid or protein metabolism, obesity, diabetes, cardiovascular disease, fibrosis, various cancers, kidney failure, immune pathologies, neurodegenerative diseases, and various monogenetic metabolic diseases described in the Online Mendelian Inheritance in Man database (Center for Medical Genetics, Johns Hopkins University (Baltimore, Md.) and National Center for Biotechnology Information, National Library of Medicine (Bethesda, Md.). In one embodiment, the condition or disorder is diabetes or obesity.

The inventors applied this new model in a study of a diabetes marker, HbA1c and identified many more vSNPs, than SNPs than would be identified with the traditional association approach. Next the inventors used genome-wide gene expression and genetic information to show that a large number of SNPs are also associated with variability in gene expression, which are designated as expression variable trait loci (eVTL). The invention provides that vSNPs for HbA1c and gene expression are highly enriched near regions in the genome that are variably methylated. Further, the inventors confirmed the existence of vSNPs for HbA1c and their correlation with DNA methylation in an independent cohort.

In various embodiments, the at least one variably methylated region (VMR) correlated with the variability genotype may be FGF3, KCNQ1, PER1 or any combination thereof. In another embodiment, the at least one variably methylated region (VMR) correlated with the variability genotype includes FGF3, KCNQ1, and PER1.

In another embodiment, the invention relates to a method of predicting risk for a condition or disorder in a subject. The method includes: (a) obtaining genotype data from a plurality of samples; (b) obtaining genome-wide gene expression data from the samples; (c) performing a first variability test for the genotype data, thereby obtaining genotype variability information; (d) performing a second variability test for at least one selected gene expression from the samples, thereby obtaining gene expression variability information, wherein the selected gene expression correlates with the condition or disorder; (e) performing a first association study between the genotype variability information of (c) and the gene expression variability information of (d), thereby identifying at least one variability genotype associated with the selected gene expression; (f) performing a second association study between each of the at least one variability genotype identified in (e) and the genome-wide gene expression data of (b), thereby identifying at least one expression variable trait loci (eVTL), wherein the at least one eVTL is associated with the condition or disorder; (g) identifying a plurality of variably methylated regions (VMRs) correlated with the selected gene expression; (h) performing a linkage disequilibrium (LD) study between the at least one variability genotype identified in (e) and the VMRs correlated with the selected gene expression identified in (g), thereby identifying at least one VMR correlated with the variability genotype; (i) measuring expression level of the at least one eVTL in (f) in a biological sample from the subject; (j) measuring methylation level of the at least one VMR correlated with the variability genotype identified in (g) in a biological sample from the subject; and (k) predicting the risk for the condition or disorder in the subject based on the expression level of the eVTL in (i) and the methylation level measured in (j).

In some embodiments, the method further includes a step of performing a third association study between the genotype data of (a) and the selected gene expression from the samples, thereby identifying at least one mean genotype associated with the selected gene expression.

The invention provides alternative sources of disease risk, that are not genetic variants for a phenotype per se, but variants for variability itself. This idea arose from the inventors' efforts to resolve the relationship between evolution, developmental biology and epigenetics, the study of non-sequence based information heritable during cell division. Previous efforts to incorporate epigenetics into evolutionary thinking have focused on Lamarckianism, i.e., epigenetic changes caused by the environment and masquerading as mutations. While examples certainly exist, it may be difficult to understand how common Lamarckian variants would be stably transmitted for the hundreds of generations necessary for evolutionary effects. Instead, the invention provides a stochastic epigenetic variation model, in which genetic variants that do not change the mean phenotype could change the variability of phenotype; and this can be mediated epigenetically. Thus, the invention provides a critical role for stochastic variation itself in natural selection. Further, the inventors identified specific variably DNA-methylated regions in isogenic mice, as well as in humans, found they are enriched for genes for development and morphogenesis, and found genetic variants, namely gain or loss of CpG dinucleotides, that helped explain the differences in differential methylation across evolution, specifically mouse and human.

The methodology of the invention makes three specific predictions for common human disease: (1) common genetic variants exist that are associated variation per se without affecting mean phenotype; (2) these variants will affect proximate genes, i.e. they are not masquerading for genetic interactions; (3) the variants are in linkage disequilibrium with genomic locations harboring variably methylated regions (VMRs). The model of the invention provides strong support for the first two predictions, and suggestive evidence for the third. As the model of the invention does not require variable DNA methylation, these data can encourage re-examination of existing GWAS data and integration into future large-scale studies.

The methodology of the invention identifies common genetic variants that are associated with phenotypic variation per se without affecting the mean phenotype. These variants are associated with the expression of proximate genes, and they are associated with variably methylated regions. These data strongly support the model of the invention for stochastic variation in phenotype that is genetically determined.

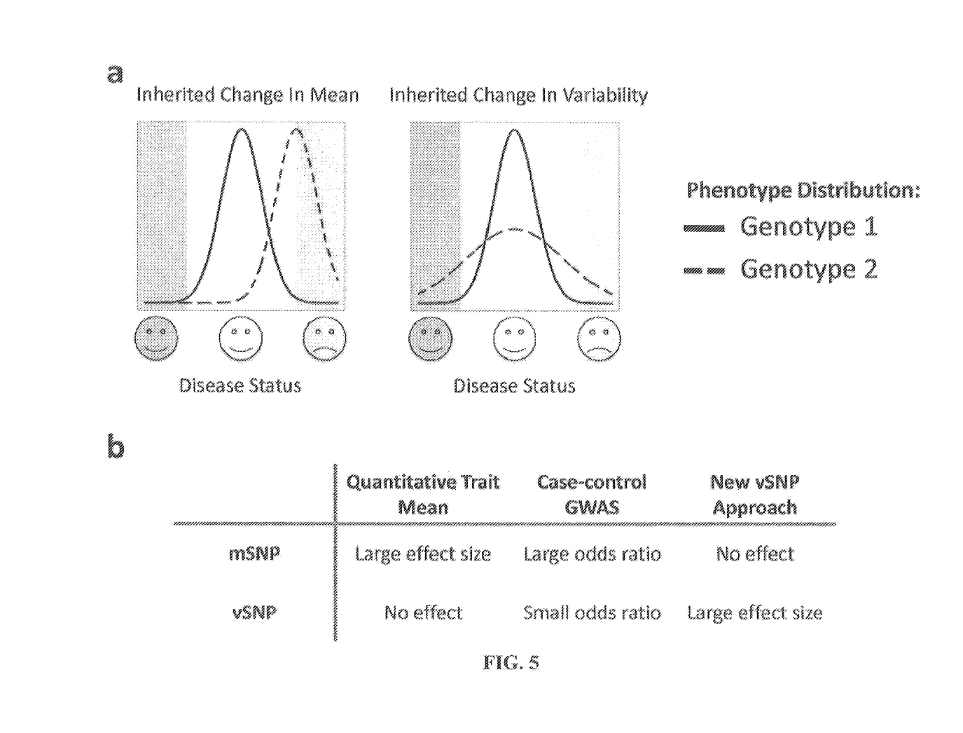

A strong mSNP would lead to a large effect size in a quantitative trait analysis and a large odds ratio in a case-control GWAS (FIG. 5), although large odds ratios in such studies have not generally been found. The invention provides that much of the variation in quantitative traits underlying common disease may be caused by genotypes that lead to increased variance per se. Individuals carrying such “variance” alleles are equally likely to lie at both the “healthy” and “diseased” spectrum of the phenotype making them difficult to identify with current GWAS approaches (FIG. 5). However, a conventional case-control GWAS analysis of such vSNPs will in fact lead to apparently small but nonzero odds ratios, since there will be some enrichment for disease status at one tail of the phenotypic spectrum (FIG. 5).

FIG. 5 shows the relationship between the new variability model and common disease. FIG. 5A is an illustration of how mSNPs and vSNPs would affect disease status through a quantitative trait. When the inheritance of an allele leads to a shift in the mean of the quantitative trait distribution, more individuals fall into the unhealthy range. When the inheritance of the allele leads to a change in variance, more individuals with that allele will be in both the unhealthy and very healthy ranges. FIG. 5B depicts the expected mSNP and vSNP effect sizes detected by quantitative trait analysis, case-control analysis, and the variance procedure of the invention. In a GWAS case-control study vSNPs may result in small but observable effects, as are frequently observed.

To test this idea, the inventors examined the enrichment of SNPs reported by GWAS in the vicinity of VMRs. These SNPs are obtained from a catalog of published GWAS SNPs (Hindorff et al. (2009) PNAS USA 106:9362-67) (on the World Wide Web at genome.gov/gwastudies). The inventors filter this list to 884 SNPs that are statistically significant after a multiple comparison correction. These GWAS SNPs are also highly enriched near VMRs. Thus many SNPs already identified by GWAS but not showing statistical significance as mSNPs may in fact be vSNPs, and the true effect size can be much greater if analyzed in the manner described here. The invention provides that identification of vSNPs will allow targeted surveillance of subpopulations carrying the “variance” alleles, i.e., those whose epigenetic and phenotypic profile, albeit stochastically arising, drives them toward illness.

In another embodiment, the invention provides a method for analyzing epigenetic information, using suitable computer software for use on a computer. The method includes: (a) performing a first variability test for genotype data obtained from a plurality of samples, thereby obtaining genotype variability information; (b) performing a second variability test for at least one selected gene expression from the samples, thereby obtaining gene expression variability information; (c) performing a first association study between the genotype variability information of (a) and the gene expression variability information of (b), thereby identifying at least one variability genotype associated with the selected gene expression; (d) performing a second association study between each of the at least one variability genotype identified in (c) and genome-wide gene expression data obtained from the samples, thereby identifying at least one expression variable trait loci (eVTL); and (e) performing a linkage disequilibrium (LD) study between the at least one variability genotype identified in (c) and a plurality of variably methylated regions (VMRs) correlated with the selected gene expression, thereby identifying at least one VMR correlated with the variability genotype.

In one embodiments, the method of the invention further includes the step of performing a third association study between the genotype data and the selected gene expression from the samples, thereby identifying at least one mean genotype associated with the selected gene expression. In another embodiment, the method of the invention further includes performing a gene ontology analysis for each of the at least one variability genotype.

As used herein, ontology analysis refers to analysis utilitizing data compiled in The Gene Ontology or GO database provided on the World Wide Web at geneontology.org. The Gene Ontology project provides an ontology of defined terms representing gene product properties. The ontology covers three domains: cellular component, the parts of a cell or its extracellular environment; molecular function, the elemental activities of a gene product at the molecular level, such as binding or catalysis; biological process, operations or sets of molecular events with a defined beginning and end, pertinent to the functioning of integrated living units: cells, tissues, organs, and organisms.

The invention further provides a system for performing any of the computational methods described herein. Generally, the system includes at least one computer readable medium having executable code with functionality for performing statistical algorithms, and at least one database storing gene related or other biological information, for example a gene database or ontology database.

As used herein, a database generally refers to a stored collection of data. Such data may relate to any number of biological phenomena, such as microarray analysis, methylation, ontology, literature, genes, proteins, expression data, SNPs, and the like. Examples of databases include The Gene Ontology, Genbank, a site maintained by the NCBI (ncbi.nlm.gov), the Kyoto Encyclopedia of Genes and Genomes (KEGG) (genome.ad.jp/kegg/), the protein database SWISS-PROT (ca.expasy.org/sprot/), the LocusLink database maintained by the NCBI (ncbi.nlm.nih.gov/˜ocus˜ink/), the Enzyme Nomenclature database maintained by G. P. Moss of Queen Mary and Westfield College in the United Kingdom (chem.qmw.ac.uk/iubmb/enzyme/). However, a variety of additional databases are known in the art and suitable for use with the present invention.

In one embodiment, the system includes functionality for identifying expression variable trait loci (eVTL) and variably methylated regions (VMRs) for predicting risk for a condition or disorder in a subject. The system may include: (a) a first variability module performing a first variability test for genotype data obtained from a plurality of samples, thereby obtaining genotype variability information; (b) a second variability module performing a second variability test for at least one selected gene expression, thereby obtaining gene expression variability information, wherein the selected gene expression correlates with the condition or disorder; (c) a first association module performing a first association study between the genotype variability information of (a) and the gene expression variability information of (b), thereby identifying at least one variability genotype associated with the selected gene expression; (d) a second association module performing a second association study between each of the at least one variability genotype identified in (c) and genome-wide gene expression data obtained from the samples, thereby identifying at least one expression variable trait loci (eVTL); and (e) a linkage disequilibrium module performing a linkage disequilibrium (LD) study between the at least one variability genotype identified in (c) and a plurality of VMRs correlated with the selected gene expression, thereby identifying at least one VMR correlated with the variability genotype.

In various embodiments, the system of the invention further includes additional modules for performing multiple analyses. For example, in one embodiment, the system includes a third association module, for example to perform a third association study between the genotype data and at least one selected gene expression from the samples. In various embodiments, the in the selected gene expression correlates with the condition or disorder. In another embodiment, the system of the invention further includes a gene ontology module performing a gene ontology analysis for each of the at least one variability genotype. Any number of additional modules may be envisioned to facility analysis of data.

Second, the present invention provides a method for predicting risk for a condition or disorder in a subject over time. Additionally, the present invention provides a method for generating an epigenetic signature for a subject which may be used, for example, to assess risk. In one instance the method is used to identify the risk of obesity. The method may also be used to target the genes involved to determine a molecular basis of the disease.

As such, the invention also relates to use of the method and system described herein to detect personalized epigenomic signatures stable over time and covarying with a phenotypic parameter of a disease or disorder of a subject. In this manner the invention provides a novel epigenetic strategy for identifying patients at risk of a common disease or disorder. In one embodiment, the parameter is a subject's body mass index (BMI).

In one embodiment, the present invention provides a method for predicting risk for a condition or disorder in a subject over time. The method includes: (a) measuring intra-sample change over time for genome-wide variably methylated regions (VMRs) from a plurality of samples; (b) performing gene ontology analysis for the VMRs; (c) identifying at least one VMR correlated with the condition or disorder using a linear regression model; (d) measuring methylation level of the at least one VMRs correlated with the condition or disorder in a biological sample from the subject; and (e) predicting the risk for the condition or disorder in the subject based on the methylation level measured in (d).

It will be understood that the steps described in any method herein may be used in combination with any other method steps described throughout this application. Further, steps of any method described herein may be used in any order.

In another embodiment, the present invention is related to a method for generating an epigenetic signature for a subject. The method includes: (a) measuring intra-sample change over time for genome-wide variably methylated regions (VMRs) from a plurality of samples; (b) separating selected VMRs into two groups using a two component Gaussian mixture model based on the measured intra-sample change of (a), wherein the VMRs in the higher distribution are designated as dynamic VMRs and the VMRs in the lower distribution are designated as stable VMRs; (c) measuring methylation levels of a plurality of stable VMRs in a biological sample from the subject; and (d) generating the epigenetic signature for the subject based on the methylation levels measured in (c).

As discussed herein, in various embodiment of the invention, the condition or disorder is body mass index (BMI), obesity or diabetes.

The epigenome consists of non-sequence-based modifications such as DNA methylation that are heritable during cell division and that may affect normal phenotypes and predisposition to disease. The inventors performed unbiased genome-scale analysis of ˜4 million CpG sites in 74 individuals using comprehensive array-based relative methylation (CHARM) analysis. The inventors found 227 regions with extreme inter-individual variability (variably methylated regions (VMRs)) across the genome, which are enriched for developmental genes based on Gene Ontology analysis. Furthermore, half of these VMRs are stable within individuals over an average of 11 years, and these VMRs define a personalized epigenomic signature. Four of these VMRs showed covariation with body mass index consistently at two study visits and are located in or near genes determined by the method herein to be implicated in regulating body weight or diabetes as discussed above.

Comprehensive Array-based Relative Methylation (CHARM) analyses were performed on samples of the AGES study, assessing 4.5 million CpG sites genome-wide, which has been shown to identify differential DNA methylation without assumptions regarding where such changes would be, and uses arrays tiled through regions based on their relative CpG content, including all CpG islands, as well as CpG island “shores” which have been shown to be enriched in differential methylation.

In brief, the AGES study constitutes visit 7 (in 2002-2005) of the Reykjavik Study, which began with 18,000 residents of Reykjavik recruited in 1967. The AGES study recruited 5758 of the surviving members, who were aged 69-96 years in 2002. Of these, 638 gave a DNA sample in 1991 as part of the sixth Reykjavik Study visit, and therefore have DNA from two time points, about 11 years apart, available for methylation analysis. The inventors present data for 74 samples, a random set of those who had ample DNA remaining for both study visits. Descriptive statistics for these samples are given in Table 1.

TABLE 1
Descriptive Information (Mean (standard error)) for
Samples Used in CHARM Analyses at Each Time Point

| | Visit 6 | Visit 7 |
|---|---|---|
| | (1991) | (2002-2005) |
| Age | 74.08 (3.49) | 82.80 (3.45) |
| Sex (% male) | 0.33 | 0.31 |
| BMI | 26.56 (3.81) | 26.01 (4.10) |
| Glucose | 0.08 (0.28) | 0.11 (0.32) |
| Type 2 diabetes (%) | 5.90 | 5.79 |
| Coronary events (%) | 0.10 | 0.14 |
| Waist/hip ratio | — | 0.66 (0.10) |
| Fat percent | — | 29.31 (7.89) |
| Hemoglobin A1C | — | 0.47 (0.07) |
| | N = 48 | N = 64 |

CHARM analysis of samples obtained from visit 7 identifies 227 regions meeting the criteria for polymorphic methylation patterns across individuals (variably methylated regions, VMRs). These represent regions of extreme variability across individuals defined by 10 or more consecutive probes with an average standard deviation>0.125 (Table 4). These VMRs show enrichment for development and morphogenesis categories (Table 2), including genes from all four HOX clusters. The appearance of developmental genes is predicted by the model of the invention that epigenetic variation would involve developmental genes, and this variability itself increases evolutionary fitness in an environmentally changing world.

TABLE 2
Gene Ontology Results with P < 0.01 for 227 VMRs Identified

| | | Odds | Obs | Expected | | |
|---|---|---|---|---|---|---|
| Pvalue | FDR | Ratio | Count | Count | GO Term | Genes |
| 0.0011 | 0.222 | 7.04 | 5 | 0.79 | Ant/post. pattern | HOXA5; HOXB6; |
| | | | | | formation | HOXD8; HOXC10; |
| | | | | | | HOXA1 |
| 0.0019 | 0.222 | 43.31 | 2 | 0.07 | blastoderm | HOXB6; HOXD8 |
| | | | | | segmentation | |
| 0.0019 | 0.222 | 43.31 | 2 | 0.07 | determ. | HOXB6; HOXD8 |
| | | | | | anterior/post. axis, | |
| | | | | | embryo | |
| 0.0082 | 0.256 | 17.31 | 2 | 0.14 | neuron recognition | FOXG1; NTM |
| 0.0086 | 0.256 | 3.63 | 6 | 1.77 | pattern | HOXA5; FOXG1; |
| | | | | | specification | LEF1; HOXC10; |
| | | | | | process | MYF6; HOXA1 |
| 0.0096 | 0.256 | 7.47 | 3 | 0.44 | placenta | ESX1; LEF1; CDX4 |
| | | | | | development | |
| 0.0096 | 0.256 | 15.74 | 2 | 0.15 | intra-Golgi vesicle- | COPZ1; |
| | | | | | mediated transport | GABARAPL2 |

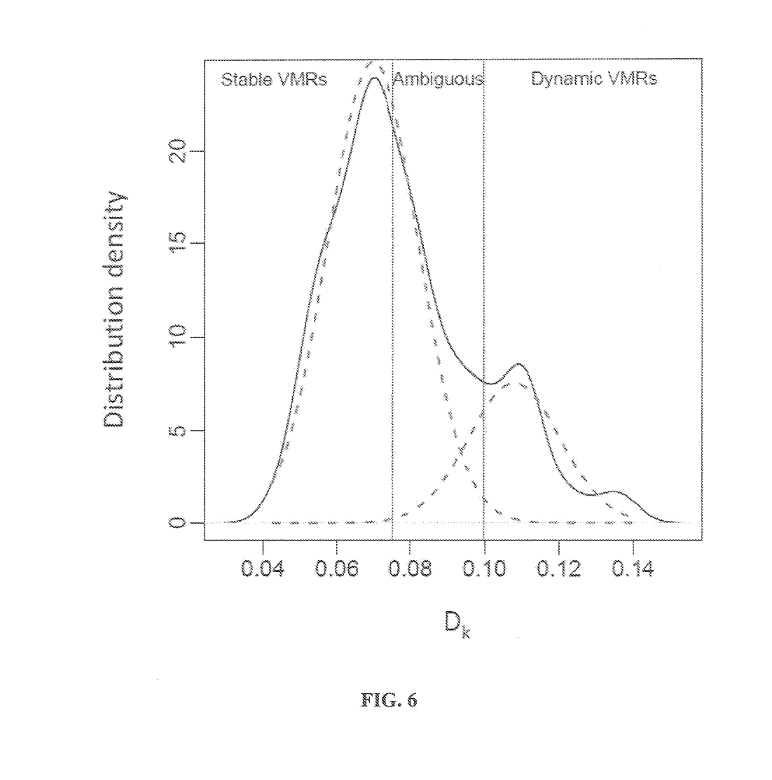

Next, to determine whether methylation at these regions changed within individuals over time, the inventors analyzed the distribution of the absolute value of average within-person change in methylation over time per VMR and found two underlying distributions (FIG. 6). This fits a two-component mixture model, with 41 VMRs easily classified into the higher intra-individual difference group (probability of membership in distribution>0.99, FIG. 6), defined as dynamic VMRs, 119 VMRs easily classified into the lower distribution (probability of green distribution>0.99), defined as stable VMRs, and 67 residing in the overlapping region labeled ambiguous, with respect to intra-individual change over time. Thus, approximately half the regions that are variably methylated across individuals appear to be stable over time within individuals.

FIG. 6 shows distribution of intra-individual change over time at VMRs. Mixture distribution analysis shows Dₖ, the average absolute value of intro-individual differences in methylation over time for VMR k, fits two underlying curves: stable showing little change and dynamic showing larger changes; ambiguous is intermediate in Dₖ.

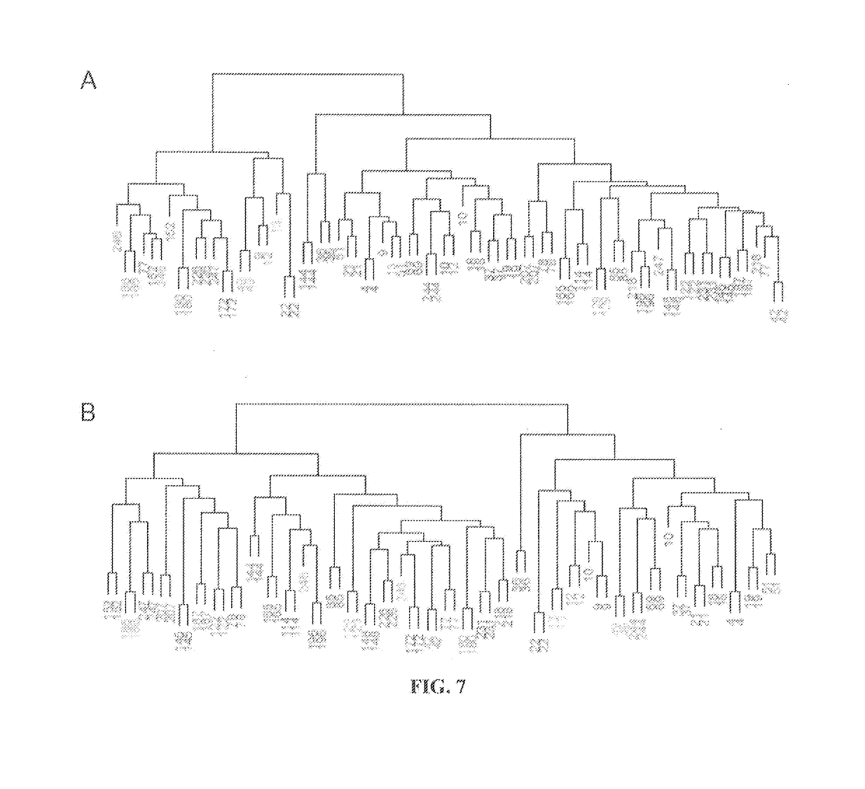

Clustering of the 227 VMR methylation profiles (FIG. 7A) revealed mixing of methylation profiles among the individuals, whereas use of only stable VMRs in the clustering algorithm uniquely identified each individual (FIG. 7B). These stable VMRs may represent polymorphic methylated regions that are not particularly susceptible to exposure modifications or that do not naturally change with age.

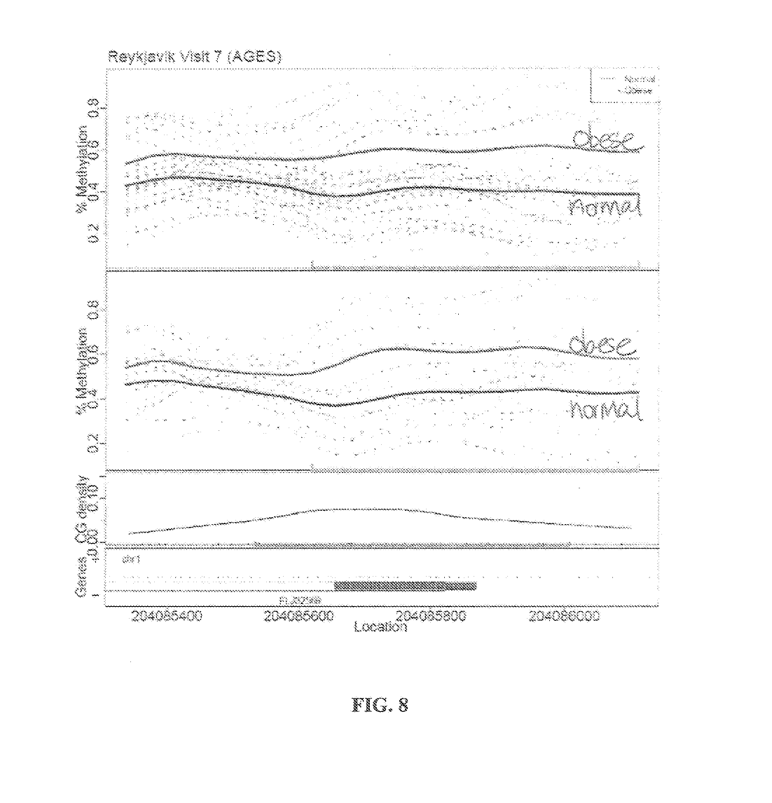

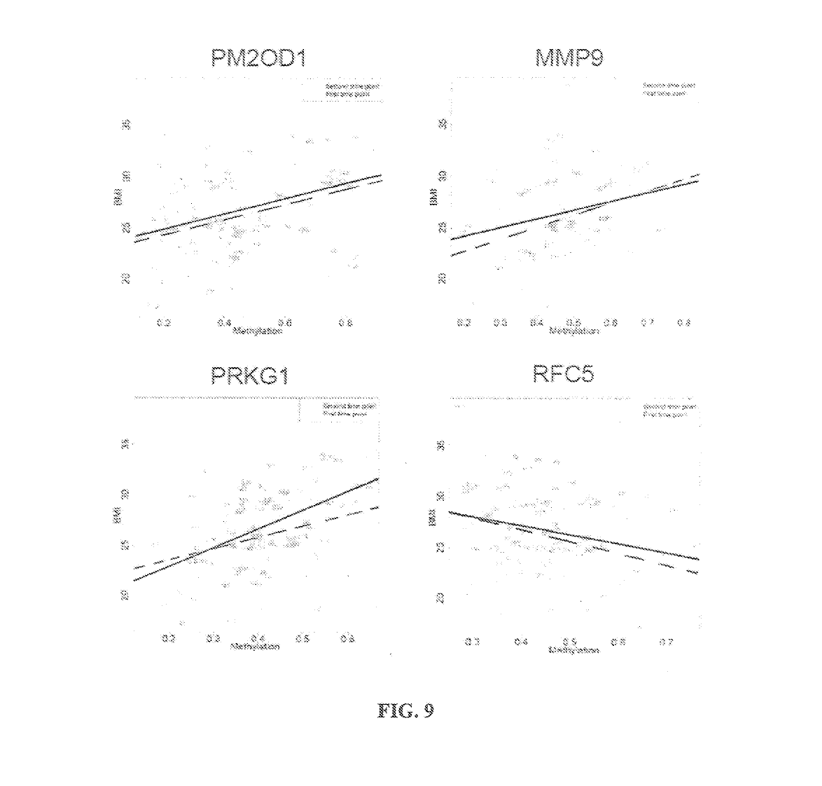

To explore how methylation of particular VMRs may play a role in disease risk, the inventors determined the relationship between methylation and BMI, an accessible and treatable phenotype that is known to have many disease correlates. The inventors identify 13 VMRs that met a false discovery rate (FDR) criteria of <25% in cross-sectional analyses of visit 7 (Table 3). Of these, 4 had a P<0.10 and the same strength and direction of correlation with BMI at the earlier visit 6. These VMRs are in or near genes PM20D1, MMP9, PRKG1, and RFC5. The methylation curves among obese (BMI·30) and normal (BMI<25) subjects for the VMR at PM20D1 illustrate approximately 20% increase in methylation that persists over time between the two visits (FIG. 8). Scatter plots for the relationship between methylation and BMI for all four VMRs exhibited significant correlations at both visits (FIG. 9).

FIG. 8 shows methylation curves for visit 7 and visit 8 data. Dashed lines are individual methylation curves. Solid lines are average curves by obese and normal groups. Bold straight lines, at the bottom of upper two boxes, indicate the boundaries of the VMR. CpG density is shown with CpG islands as a bold straight line at the bottom of the third box from the top. Gene location shown at bottom.

FIG. 9 shows correlation between methylation and BMI at six BMI-related VMRs. Points are individual IDs. Solid lines indicate visit 6 (first visit), and dotted lines indicate visit 7 (second visit).

The methodology of the invention determines global DNA methylation changes within individuals over time as well as the locations of site-specific changes at dynamic VMRs using a genome-wide approach. In addition, the invention provides a separate set of stable VMRs that can be used to uniquely identify individuals, in an epigenetic signature akin to genetic fingerprinting. This signature may be correlated with disease status, implying that an epigenetic signature can mark disease risk or disease states.

In one embodiment, the invention provides stable VMRs that correlate with BMI at least two separate visits a decade apart.

Some have argued that DNA methylation changes over time and is an important biological mediator of environmental effects on human disease, while others support the concept of inherited DNA methylation patterns, implying they are potentially variable across individuals but less likely to be dynamic over time. This has been a conundrum, since these appear to be opposing ideas. However, the inventors showed that both ideas have merit. It is important to identify these regions in the context of disease consequences, since those that are particularly labile may be the sites relevant when considering epigenetic marks as mediators of environmental effects, while those that are stable may be relevant as mediators or moderators of genetic effects. Further, those that do not change over time can be used as an epigenetic signature for and individual, similar to genotype. These regions can then be considered as candidates for assessment of methylation associations with disease or health-related phenotypes under specific risk models.

TABLE 3
Stable VMRs Associated with BMI

| | | Visit 7 | | | Visit 6 | |
|---|---|---|---|---|---|---|
| | | | | Regres- | | Regres- |
| | Nearest | | | sion | | sion |
| Chr | Gene | Qval | Pval | Estimate | Pval | Estimate |
| chrX | IL1RAPL2 | 0.114 | 0.00304 | −20.3 | 0.266 | −8.9 |
| chr1 | PM2OD1 | 0.114 | 0.00332 | 7.6 | 0.00824 | 7.7 |
| chr6 | NEDD9 | 0.114 | 0.00351 | 12.1 | 0.38 | 5.2 |
| chr20 | MMP9 | 0.160 | 0.00658 | 11.6 | 0.0605 | 8.9 |
| chr10 | SORCS1 | 0.215 | 0.0128 | −13.6 | 0.112 | −9.4 |
| chr10 | PRKG1 | 0.215 | 0.0132 | 11.8 | 0.000711 | 18.9 |
| chr12 | RFC5 | 0.243 | 0.0175 | −11.8 | 0.0653 | −8.8 |
| chr1 | TTC13 | 0.249 | 0.022 | 9.27 | 0.523 | 3.3 |
| chrX | DACH2 | 0.249 | 0.0311 | −15.1 | 0.539 | 4.1 |
| chr5 | TRIM36 | 0.249 | 0.0326 | 11.3 | 0.0781 | −14.1 |
| chr14 | FLRT2 | 0.249 | 0.0278 | −9.5 | 0.19 | −5.8 |
| chr1 | C1orf57 | 0.249 | 0.0253 | −10.6 | 0.282 | −6.5 |
| chr18 | APCDD1 | 0.249 | 0.0332 | −10.7 | 0.901 | 0.7 |

Bold values indicate confirmation in visit 6 analysis (p < 0.1 and consistent regression parameter estimates); italics indicate conflicting directions of correlation with BMI

The invention helps to focus the integration of methylation measurement into epidemiologic studies of disease risk by providing specific genomic sites for inquiry. The exploration of possible correlations between methylation at these VMRs and an easily measured disease-related phenotype, BMI, identified 13 genes, 4 of which are consistently correlated with BMI across two separate study visits. Many of these 13 genes have been previously implicated in obesity or diabetes. MMP9, as well as another member of this family, MMP3, encode a metallopeptidase that is upregulated in obese individuals. Several MMPs, including MMP9, are known to be upregulated in human adipocytes. Matrix metallopeptidases have also been associated with obesity in rodent models. Interestingly, PM20D1 is also a metalloproteinase and, although not yet well-characterized, may have similar implications for obesity. PRKG1, a cGMP-dependent protein kinase, plays an important role in foraging behavior, food acquisition and energy balance. RFC5 is an intriguing gene as it encodes a metabolism-linked DNA replication complex loading protein, dysfunction of which leads to DNA repair defects. It might thus play a role in well-known but poorly understood DNA damage related complications of diabetes.

In one embodiment, the at least one VMR correlated with the condition or disorder is selected from MMP9, PRKG1, RFC5, CACNA2D3, PM20D1 or any combination thereof. In one embodiment, the at least one VMR correlated with the condition or disorder includes MMP9, PRKG1, RFC5, CACNA2D3, and PM20D1. In another embodiment, the at least one VMR correlated with the condition or disorder has at least one nearest gene selected from IL1RAPL2, PM2OD1, NEDD9, MMP9, SORCS1, PRKG1, RFC5, TTC13, DACH2, TRIM36, FLRT2, C1orf57, and APCDD1. In an additional or alternative embodiment, IL1RAPL2, PM2OD1, NEDD9, MMP9, SORCS1, PRKG1, RFC5, TTC13, DACH2, TRIM36, FLRT2, C1orf57, APCDD1 or combination thereof are nearest genes to the at least one VMR correlated with the condition or disorder.

In an obese mouse model, SORCS1 has been located at a type 2 diabetes quantitative trait locus (QTL), and this has been confirmed in humans, where SORCS1 SNPs and haplotypes were associated with fasting insulin secretion. IL1RAPL2 is located at a region on chromosome X that is associated with Prader-Willi like syndrome, while DACH2 is also an X-linked gene associated with Wilson-Turner syndrome, both of which are Mendelian disorders with obesity features. TTC13 is part of a family containing another tetratricopeptide repeat gene, TTC8, that has been directly linked to Bardet-Biedl syndrome, which includes obesity as a primary feature. APCDD1 is a positional candidate gene associated with QTL that affects fat deposition in pigs and is located at a region on chromosome 18 that is linked to percentage of body fat in men.

The identification of VMRs is of course limited by the number of individuals contributing to a particular genome-wide CHARM analysis. It is likely that increased sample sizes improve detection of additional VMRs. Further, the dynamic VMRs defined here are based on an eleven year window among elderly participants. It is important to also identify methylomic regions that show intra-individual changes at early segments of the lifespan and to connect these changes to particular environmental exposures. One potential caveat from these analyses is that the methylation patterns are obtained from DNA derived from blood, and thus contain a mixture of cell types that can confound the results. However, in a previous study of global DNA methylation (i.e., non-site-specific) in these samples, no relationship was found between lymphocyte count and methylation. Cellular heterogeneity may not be associated with DNA methylation amounts for the majority of sites they studied. The use of blood as a DNA source may also limit the interpretations of these results, given the tissue specificity of DNA methylation. However, there is growing precedent for lymphoid tissues serving as a good surrogate tissue for changes in other target tissues. For example, loss of imprinting (LOI) of IGF2, one of the best studied disease-related epigenetic mutations, is found in both lymphocytes and colon, and changes of either are associated with increased colorectal cancer risk. Further, the exploration of the correlation between BMI and methylation was based on availability of quantitative data and relevance to human disease. One may be unable to assess the relationship of VMRs to categorical outcomes in this sample that is, although more comprehensive than previous genome-wide site-specific methylation reports, the sample number limited the analysis to relationship between methylation and quantitative phenotype, rather than categorical outcomes. The invention provides further examination of other measures of obesity, and to disease outcomes such as diabetes and cardiovascular disease, with respect to the particular VMRs identified here.

The implications of these results are wide-ranging. An individual epigenetic signature that is stable over time has not previously been described. Such a signature could be driven by underlying sequence variation, by early environmental exposure, e.g. prenatally, or both. These stable VMRs would likely complement genotype, because they would also reflect early exposure. In addition, the invention provides that some genetic variants would drive increasing site-specific stochastic epigenetic variation, and thus the variance of methylation in a population could be predicted by genotype, the methylation level in an individual would not be predictable from genotype and would require direct measurement.

Even if in part or completely genetically driven, this epigenotype may be more proximate to the ultimate phenotype, in this case body mass index, and thus have considerable value for disease risk assessment. Although the sample size is larger than previous genome-scale gene-specific methylation studies, it is still relatively small compared to classical sequence-driven approaches such as GWAS. Even so, the data suggest that this epigenomic approach to disease phenotype will be an important complement to such studies. Given the restraint of relatively small sample numbers, the inventors can identify at least four genes with VMRs related to BMI. In addition, the identification of stable VMRs may have long term consequences for developing personalized epigenomics in medicine, with the hope of forging a connection that accurately reflects personal genomes with early (e.g., in utero) environments.

While the present invention exemplifies the CHARM assay for detection of methylation, in fact numerous methods for analyzing methylation status of a DNA are known in the art and can be used in the methods of the present invention to identify methylation status. In various embodiments, the determining of methylation status in the methods of the invention is performed by one or more techniques selected from the group consisting of a nucleic acid amplification, polymerase chain reaction (PCR), methylation specific PCR, bisulfite pyrosequencing, single-strand conformation polymorphism (SSCP) analysis, restriction analysis, microarray technology, and proteomics. Analysis of methylation can be performed by bisulfite genomic sequencing. Bisulfite treatment modifies DNA converting unmethylated, but not methylated, cytosines to uracil. Bisulfite treatment can be carried out using the METHYLEASY bisulfite modification kit (Human Genetic Signatures).

In some embodiments, bisulfite pyrosequencing, which is a sequencing-based analysis of DNA methylation that quantitatively measures multiple, consecutive CpG sites individually with high accuracy and reproducibility, may be used.

Altered methylation can be identified by identifying a detectable difference in methylation. For example, hypomethylation can be determined by identifying whether after bisulfite treatment a uracil or a cytosine is present a particular location. If uracil is present after bisulfite treatment, then the residue is unmethylated. Hypomethylation is present when there is a measurable decrease in methylation.

In an alternative embodiment, the method for analyzing methylation status can include amplification using a primer pair specific for methylated residues within a VMR. In these embodiments, selective hybridization or binding of at least one of the primers is dependent on the methylation state of the target DNA sequence (Herman et al., Proc. Natl. Acad. Sci. USA, 93:9821 (1996)). For example, the amplification reaction can be preceded by bisulfite treatment, and the primers can selectively hybridize to target sequences in a manner that is dependent on bisulfite treatment. For example, one primer can selectively bind to a target sequence only when one or more base of the target sequence is altered by bisulfite treatment, thereby being specific for a methylated target sequence.

Other methods are known in the art for determining methylation status of a VMR, including, but not limited to, array-based methylation analysis and Southern blot analysis.

Methods using an amplification reaction, for example methods above for detecting hypomethylation or hypermethylation of one or more VMRs, can utilize a real-time detection amplification procedure. For example, the method can utilize molecular beacon technology (Tyagi et al., Nature Biotechnology, 14: 303 (1996)) or Taqman™ technology (Holland et al., Proc. Natl. Acad. Sci. USA, 88:7276 (1991)).

Also methyl light (Trinh et al., Methods 25(4):456-62 (2001), incorporated herein in its entirety by reference), Methyl Heavy (Epigenomics, Berlin, Germany), or SNuPE (single nucleotide primer extension) (see e.g., Watson et al., Genet Res. 75(3):269-74 (2000)) Can be used in the methods of the present invention related to identifying altered methylation of VMRs.

The degree of methylation in the DNA associated with the VMRs being assessed, may be measured by fluorescent in situ hybridization (FISH) by means of probes which identify and differentiate between genomic DNAs, associated with the VMRs being assessed, which exhibit different degrees of DNA methylation. FISH is described, for example, in de Capoa et al. (Cytometry. 31:85-92, 1998) which is incorporated herein by reference. In this case, the biological sample will typically be any which contains sufficient whole cells or nuclei to perform short term culture. Usually, the sample will be a sample that contains 10 to 10,000, or, for example, 100 to 10,000, whole cells.

Additionally, as mentioned above, methyl light, methyl heavy, and array-based methylation analysis can be performed, by using bisulfite treated DNA that is then PCR-amplified, against microarrays of oligonucleotide target sequences with the various forms corresponding to unmethylated and methylated DNA.

The term “nucleic acid molecule” is used broadly herein to mean a sequence of deoxyribonucleotides or ribonucleotides that are linked together by a phosphodiester bond. As such, the term “nucleic acid molecule” is meant to include DNA and RNA, which can be single stranded or double stranded, as well as DNA/RNA hybrids. Furthermore, the term “nucleic acid molecule” as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR), and, in various embodiments, can contain nucleotide analogs or a backbone bond other than a phosphodiester bond.

The terms “polynucleotide” and “oligonucleotide” also are used herein to refer to nucleic acid molecules. Although no specific distinction from each other or from “nucleic acid molecule” is intended by the use of these terms, the term “polynucleotide” is used generally in reference to a nucleic acid molecule that encodes a polypeptide, or a peptide portion thereof, whereas the term “oligonucleotide” is used generally in reference to a nucleotide sequence useful as a probe, a PCR primer, an antisense molecule, or the like. Of course, it will be recognized that an “oligonucleotide” also can encode a peptide. As such, the different terms are used primarily for convenience of discussion.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

In another embodiment, the present invention includes kits that are useful for carrying out the methods of the present invention. The components contained in the kit depend on a number of factors, including: the particular analytical technique used to detect methylation or measure the degree of methylation or a change in methylation, and the one or more VMRs is being assayed for methylation status.

In another embodiment, the present invention provides a kit for detecting risk of a condition or disorder. The kit includes a plurality of oligonucleotide primer sequences capable of generating a plurality of amplificates from genomic DNA, the amplificates including variably methylated region (VMR) sequences as set forth in Table 4, and any combination thereof. The kit may further include instructions for detecting risk. In one embodiment, the condition or disorder is diabetes or obesity. In a related embodiment, the kit may further include computer executable code and instructions for performing statistical analysis.

Accordingly, the present invention provides a kit for determining a methylation status of one or more VMRs of the invention. In some embodiments, the one or more VMRs are selected from one or more of the sequences as set forth in Table 4. The kit includes an oligonucleotide probe, primer, or primer pair, or combination thereof for carrying out a method for detecting methylation status, as discussed above. For example, the probe, primer, or primer pair, can be capable of selectively hybridizing to the DMR either with or without prior bisulfite treatment of the DMR. The kit can further include one or more detectable labels.

The kit can also include a plurality of oligonucleotide probes, primers, or primer pairs, or combinations thereof, capable of selectively hybridizing to the DMR with or without prior bisulfite treatment of the DMR. The kit can include an oligonucleotide primer pair that hybridizes under stringent conditions to all or a portion of the DMR only after bisulfite treatment. The kit can include instructions on using kit components to identify, for example, the increased risk of developing diabetes or obesity.

As used herein, the term “selective hybridization” or “selectively hybridize” refers to hybridization under moderately stringent or highly stringent physiological conditions, which can distinguish related nucleotide sequences from unrelated nucleotide sequences.

As known in the art, in nucleic acid hybridization reactions, the conditions used to achieve a particular level of stringency will vary, depending on the nature of the nucleic acids being hybridized. For example, the length, degree of complementarity, nucleotide sequence composition (for example, relative GC:AT content), and nucleic acid type, for example, whether the oligonucleotide or the target nucleic acid sequence is DNA or RNA, can be considered in selecting hybridization conditions. An additional consideration is whether one of the nucleic acids is immobilized, for example, on a filter. Methods for selecting appropriate stringency conditions can be determined empirically or estimated using various formulas, and are well known in the art (see, e.g., Sambrook et al., supra, 1989).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, for example, high stringency conditions, or each of the conditions can be used, for example, for 10 to 15 minutes each, in the order listed above, repeating any or all of the steps listed.

Third, the invention also relates to stochastic epigenetic variation as a driving force of development, evolutionary adaptation, and disease. Neo-Darwinian evolutionary theory is based on exquisite selection of phenotypes caused by small genetic variations, which is the basis of quantitative trait contribution to phenotype and disease. Epigenetics is the study of nonsequence-based changes, such as DNA methylation, heritable during cell division. Previous attempts to incorporate epigenetics into evolutionary thinking have focused on Lamarckian inheritance, that is, environmentally directed epigenetic changes. Provided is a new non-Lamarckian theory for a role of epigenetics in evolution. The inventors suggest that genetic variants that do not change the mean phenotype could change the variability of phenotype; and this could be mediated epigenetically. This inherited stochastic variation model would provide a mechanism to explain an epigenetic role of developmental biology in selectable phenotypic variation, as well as the largely unexplained heritable genetic variation underlying common complex disease.

Two experimental results are provided as proof of principle. The first result is direct evidence for stochastic epigenetic variation, identifying highly variably DNA-methylated regions in mouse and human liver and mouse brain, associated with development and morphogenesis. The second is a heritable genetic mechanism for variable methylation, namely the loss or gain of CpG dinucleotides over evolutionary time. Further, the inventors modeled genetically inherited stochastic variation in evolution, showing that it provides a powerful mechanism for evolutionary adaptation in changing environments that can be mediated epigenetically. These data suggest that genetically inherited propensity to phenotypic variability, even with no change in the mean phenotype, substantially increases fitness while increasing the disease susceptibility of a population with a changing environment.

These results provide a basis for another embodiment of the invention. In one embodiment, the invention provides to a method for simulating epigenetic plasticity across generations. The method includes: (a) generating a plurality of genotype variants, wherein the genotype variants are genetically inherited; (b) applying natural selection favoring a first subset of the genotype variants; (c) enabling a plurality of stochastic epigenetic elements, wherein the stochastic epigenetic elements change phenotypes without changing the genotype variants; (d) allowing a changing environment across generations favoring a second subset of the genotype variants; and (e) monitoring fluctuations of mean phenotype across generations.

In one embodiment, the method of the invention further includes comparing frequency of fitness from genome-wide association study (GWAS) with the genotype variants which change the mean phenotype.

A variety of statistical models may be used with the methods of the invention. In one embodiment, a Fisher-Wright neutral selection model is used. In another embodiment, a Fisher's additive model is used. In another embodiment, a multinomial distribution is used. In another embodiment, each of the genotype variants has two possible polymorphisms. In another embodiment, the stochastic epigenetic elements represent additions or deletions of CpG islands.

The present invention provides an advance over Darwinism; stochastic variation, not Lamarckian Inheritance. Increased variability with a given genotype might itself increase fitness. This could arise by genetic variants that do not change the mean phenotype but do change the variability of phenotype. A natural mechanism to use to consider such a model is epigenetic plasticity during development, for example, varying DNA methylation patterns. This idea differs from Lamarckian inheritance, in that in the model of the invention the genetic change is inherited, and this change leads to increased epigenetic variation. It also differs from the likely role of epigenetics in modifying mutation rate, both through C to T transition due to deamination of methylcytosine and through modified rates of chromosomal rearrangement. The invention provides genome-scale analysis of DNA methylation in human and mouse tissues and explored them in two new ways. First, the inventors investigated whether there were regions of variable methylation across individuals for a given tissue type. Second, the inventors explore whether tissue-specific differentially methylated regions (T-DMRs) differed across species and whether the underlying DNA sequence can account for these differences.

To assess the degree of intrinsic variability in DNA methylation of a given tissue, the inventors set out to identify the location of the most highly variable regions of DNA methylation in mouse liver from four individuals. The inventors chose this specific tissue because it is relatively homogeneous. The inventors examined newborns in whom polyploidy is minimal, although copy number would not be expected to affect DNA methylation, because the method of the invention controls for copy number. Environmental effects were minimized by examining inbred mice (indeed, littermates from the same cage). Surprisingly, many loci throughout the genome showed striking variations in DNA methylation, which the inventors term variably methylated regions (VMRs). Surprisingly, these VMRs were significantly enriched in the vicinity of genes with Gene Ontogeny (GO) functional categories for development and morphogenesis (Table 5) when using either all genes for comparison or all regions present on the CHARM array, indicating that enrichment is not explained solely by high CpG content, because the array itself is designed to assay high-CpG regions.

TABLE 5
Enrichment scores of GO categories of genes in the vicinity of VMRs in mouse liver.

| GOBPID | P value | Odds ratio | Expected count | Count | Size | Term |
|---|---|---|---|---|---|---|
| GO:0048699 | 2.8E−05 | 2.0 | 26.9 | 49 | 384 | Generation of neurons |
| GO:0009880 | 8.5E−05 | 4.9 | 2.8 | 11 | 41 | Embryonic pattern specification |
| GO:0030030 | 0.00033 | 2.0 | 19.1 | 35 | 272 | Cell projection organization |
| GO:0021517 | 0.00034 | 8.8 | 1.0 | 6 | 15 | Ventral spinal cord development |
| GO:0035107 | 0.00041 | 2.9 | 6.2 | 16 | 89 | Appendage morphogenesis |
| GO:0048666 | 0.00046 | 2.0 | 17.2 | 32 | 245 | Neuron development |
| GO:0032990 | 0.00050 | 2.2 | 12.3 | 25 | 175 | Cell part morphogenesis |
| GO:0009887 | 0.00052 | 1.6 | 35.9 | 56 | 512 | Organ morphogenesis |
| GO:0021515 | 0.00055 | 6.2 | 1.5 | 7 | 22 | Cell differentiation in spinal cord |
| GO:0048812 | 0.00065 | 2.2 | 11.8 | 24 | 168 | Neurite morphogenesis |
| GO:0060173 | 0.00068 | 2.7 | 6.5 | 16 | 93 | Limb development |
| GO:0007411 | 0.00075 | 2.8 | 5.9 | 15 | 85 | Axon guidance |
| GO:0006270 | 0.00088 | 9.5 | 0.8 | 5 | 12 | DNA replication initiation |
| GO:0001708 | 0.0010 | 4.6 | 2.1 | 8 | 31 | Cell fate specification |
| GO:0000904 | 0.0014 | 2.0 | 13.2 | 25 | 188 | Cell morphogenesis involved in differentiation |
| GO:0048869 | 0.0017 | 1.3 | 86.5 | 112 | 1,231 | Cellular developmental process |
| GO:0007420 | 0.0020 | 1.9 | 15.0 | 27 | 214 | Brain development |
| GO:0048663 | 0.0021 | 3.6 | 2.9 | 9 | 42 | Neuron fate commitment |
| GO:0042415 | 0.0031 | 19.9 | 0.3 | 3 | 5 | Norepinephrine metabolic process |
| GO:0009954 | 0.0033 | 4.9 | 1.5 | 6 | 22 | Proximal/distal pattern formation |
| GO:0042472 | 0.0033 | 3.1 | 3.7 | 10 | 53 | Inner ear morphogenesis |
| GO:0048598 | 0.0035 | 1.7 | 19.4 | 32 | 277 | Embryonic morphogenesis |
| GO:0007417 | 0.0050 | 2.9 | 3.9 | 10 | 57 | Central nervous system development |
| GO:0021846 | 0.0053 | 7.6 | 0.7 | 4 | 11 | Cell proliferation in forebrain |
| GO:0021520 | 0.0058 | 13.2 | 0.4 | 3 | 6 | Spinal cord motor neuron cell fate specification |
| GO:0021521 | 0.0058 | 13.2 | 0.4 | 3 | 6 | Ventral spinal cord interneuron specification |
| GO:0045773 | 0.0058 | 13.2 | 0.4 | 3 | 6 | Positive regulation of axon extension |
| GO:0021536 | 0.0065 | 4.2 | 1.7 | 6 | 25 | Diencephalon development |
| GO:0035116 | 0.0067 | 5.1 | 1.2 | 5 | 18 | Embryonic hindlimb morphogenesis |
| GO:0007275 | 0.0076 | 1.2 | 124.8 | 149 | 1,776 | Multicellular organismal development |
| GO:0007423 | 0.0076 | 1.8 | 13.4 | 23 | 191 | Sensory organ development |
| GO:0030326 | 0.0090 | 2.6 | 4.2 | 10 | 61 | Embryonic limb morphogenesis |
| GO:0035270 | 0.0095 | 2.7 | 3.6 | 9 | 52 | Endocrine system development |
| GO:0006268 | 0.0097 | 9.9 | 0.49 | 3 | 7 | DNA unwinding during replication |
| GO:0021546 | 0.0097 | 9.9 | 0.49 | 3 | 7 | Rhombomere development |
| GO:0048856 | 0.0099 | 1.2 | 106.1 | 128 | 1,538 | Anatomical structure development |

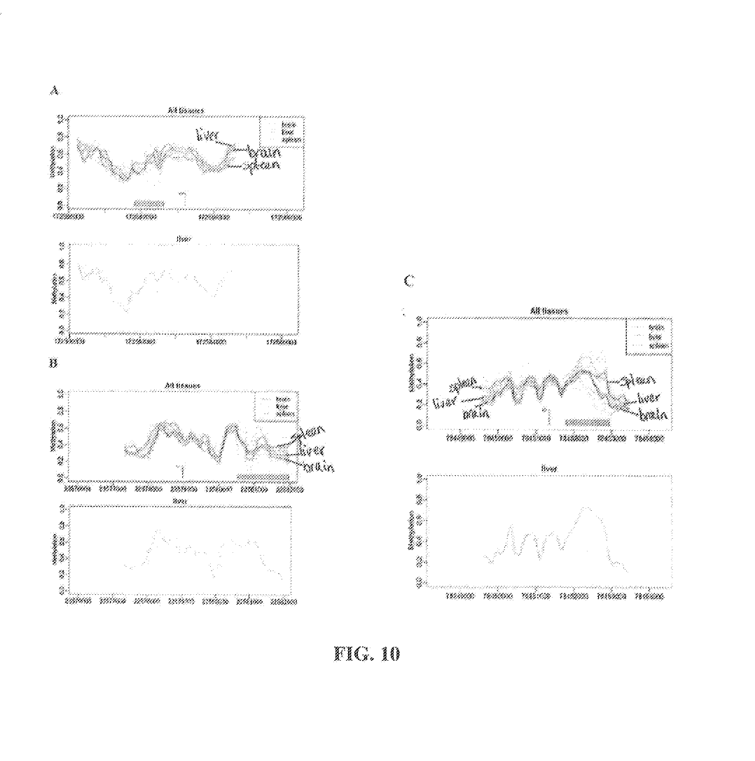

Examples of developmental genes with VMRs include: Bmp7, involved in early embryogenic programming and bone induction, Pou3f2, involved in neurogenesis and stem cell reprogramming, and Ntrk3, involved in body position sensing—are shown in FIG. 10. FIG. 10 shows examples of developmental genes with VMRs in livers from isogenic mice raised in the same environment. Shown are Bmp7 (FIG. 10A), Pou3f2 (FIG. 10B), and Ntrk3 (FIG. 10C), involved in early embryogenic programming and bone induction, neurogenesis and stem cell reprogramming, and body position sensing, respectively. In each paired plot, the top panel shows estimated methylation levels from various biological replicates from three different tissues: brain, liver, and spleen (dashed lines). The thicker solid lines represent the average curves for each tissue. The bars denote the regions in which the statistical method detected a VMR. The bottom panel highlights the liver. Only the four liver curves are shown. The different line types and colors represent the four individual mice.

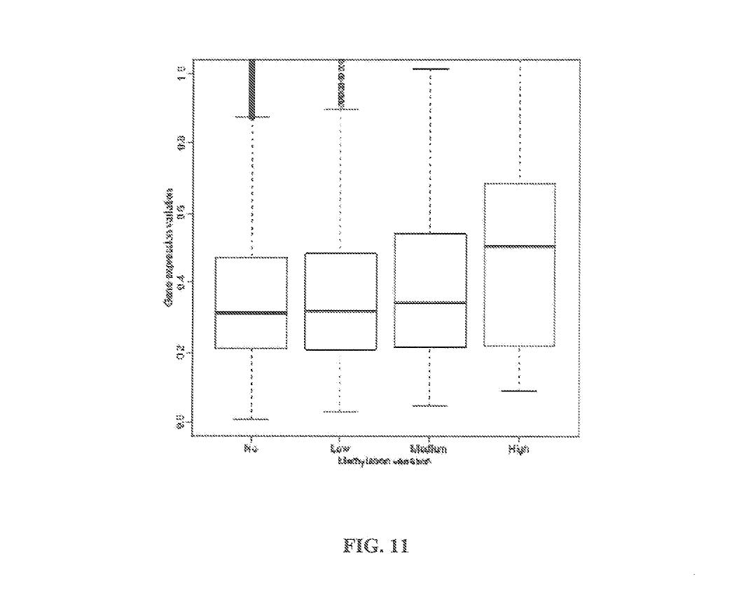

Furthermore, the VMRs are associated with a functional property: expression. As shown in FIG. 11, VMRs within 500 bp of a transcriptional start site (TSS) can exhibit a stronger association between gene expression variability and methylation variability. FIG. 11 shows VMRs being associated with variability in gene expression of nearby genes. The human liver VMRs detected with the statistical algorithm of the invention are divided into three types: low variation (lowest 70%), high variation (highest 5%), and medium variation (the remainder). The VMRs within 500 bases from a gene's transcription start site are associated with that gene. The expression measurements are obtained for the same human livers, and the SD across subjects is used to quantify variability. These boxplots show the distribution of this variability stratified by VMR variability. The first boxplot represents genes not associated with a VMR.

Human livers were examined for the presence of VMRs. Similar to the mouse results, significant variability can be found. Where the VMRs are near genes, as in the mouse, there is a strong enrichment in the vicinity of genes with GO functional categories for development and morphogenesis when controlled for the mouse CHARM array (Table 6).

TABLE 6
Enrichment scores of GO categories of genes in the vicinity of VMRs in human liver.

| GOBPID | P value | Odds ratio | ExpCount | Count | Size | Term |
|---|---|---|---|---|---|---|
| GO:0009790 | 1.8E−05 | 1.8 | 43.1 | 70 | 320 | Embryonic development |
| GO:0019222 | 2.3E−05 | 1.3 | 319.5 | 379 | 2,372 | Regulation of metabolic process |
| GO:0006355 | 4.0E−05 | 1.3 | 239.6 | 292 | 1,779 | Regulation of transcription, DNA-dependent |
| GO:0032774 | 5.0E−05 | 1.3 | 246.8 | 299 | 1,832 | RNA biosynthetic process |
| GO:0009887 | 5.3E−05 | 1.6 | 54.1 | 82 | 402 | Organ morphogenesis |
| GO:0048704 | 8.4E−05 | 4.0 | 5.2 | 15 | 39 | Embryonic skeletal system morphogenesis |
| GO:0001501 | 8.5E−05 | 1.9 | 27.8 | 48 | 207 | Skeletal system development |
| GO:0051093 | 8.5E−05 | 1.7 | 43.5 | 68 | 323 | Negative regulation of developmental process |
| GO:0016339 | 0.00012 | 7.2 | 2.2 | 9 | 17 | Calcium-dependent cell-cell adhesion |
| GO:0009952 | 0.00013 | 2.5 | 12.3 | 26 | 92 | Anterior/posterior pattern formation |
| GO:0048518 | 0.00017 | 1.3 | 133.2 | 171 | 989 | Positive regulation of biological process |
| GO:0019219 | 0.00025 | 1.2 | 269.0 | 317 | 1,997 | Regulation of nucleobase, nucleoside, nucleotide |
| | | | | | | and nucleic acid metabolic process |
| GO:0007389 | 0.00028 | 2.0 | 22.3 | 39 | 166 | Pattern specification process |
| GO:0010468 | 0.00029 | 1.2 | 272.3 | 320 | 2,029 | Regulation of gene expression |
| GO:0043009 | 0.00032 | 2.1 | 18.7 | 34 | 140 | Chordate embryonic development |
| GO:0031326 | 0.00037 | 1.2 | 279.8 | 327 | 2,077 | Regulation of cellular biosynthetic process |
| GO:0006350 | 0.00038 | 1.2 | 267.6 | 314 | 1,987 | Transcription |
| GO:0001824 | 0.00040 | 4.9 | 3.0 | 10 | 23 | Blastocyst development |
| GO:0010556 | 0.00048 | 1.2 | 271.3 | 317 | 2,014 | Regulation of macromolecule biosynthetic process |
| GO:0050678 | 0.00051 | 3.6 | 4.8 | 13 | 36 | Regulation of epithelial cell proliferation |
| GO:0048863 | 0.00064 | 7.5 | 1.7 | 7 | 13 | Stem cell differentiation |
| GO:0019827 | 0.00076 | 9.6 | 1.3 | 6 | 10 | Stem cell maintenance |
| GO:0007399 | 0.00080 | 1.4 | 84.5 | 112 | 631 | Nervous system development |
| GO:0000165 | 0.00089 | 2.0 | 16.0 | 29 | 119 | MAPKKK cascade |
| GO:0043284 | 0.0011 | 1.2 | 327.0 | 372 | 2,428 | Biopolymer biosynthetic process |
| GO:0043583 | 0.0014 | 2.7 | 7.2 | 16 | 54 | Ear development |
| GO:0042472 | 0.0016 | 3.5 | 4.1 | 11 | 31 | Inner ear morphogenesis |
| GO:0048468 | 0.0016 | 1.4 | 62.6 | 85 | 465 | Cell development |
| GO:0007420 | 0.0017 | 1.8 | 21.2 | 35 | 158 | Brain development |
| GO:0034645 | 0.0017 | 1.2 | 346.4 | 390 | 2,572 | Cellular macromolecule biosynthetic process |
| GO:0001656 | 0.0018 | 3.8 | 3.6 | 10 | 27 | Metanephros development |
| GO:0035239 | 0.0018 | 2.6 | 7.4 | 16 | 55 | Tube morphogenesis |
| GO:0043066 | 0.0019 | 1.7 | 26.9 | 42 | 200 | Negative regulation of apoptosis |
| GO:0045747 | 0.002 | Inf | 0.4 | 3 | 3 | Positive regulation of Notch signaling pathway |
| GO:0045597 | 0.0027 | 1.9 | 15.6 | 27 | 116 | Positive regulation of cell differentiation |
| GO:0043067 | 0.0030 | 1.4 | 58.7 | 79 | 436 | Regulation of programmed cell death |
| GO:0032501 | 0.0037 | 1.2 | 297.8 | 336 | 2,211 | Multicellular organismal process |
| GO:0007156 | 0.0039 | 1.9 | 13.7 | 24 | 102 | Homophilic cell adhesion |
| GO:0021546 | 0.0039 | 12.8 | 0.8 | 4 | 6 | Rhombomere development |
| GO:0065007 | 0.0040 | 1.1 | 633.7 | 677 | 4,704 | Biological regulation |
| GO:0045884 | 0.0043 | 5.5 | 1.7 | 6 | 13 | Regulation of survival gene product expression |
| GO:0048523 | 0.0043 | 1.2 | 129.7 | 157 | 963 | Negative regulation of cellular process |
| GO:0021915 | 0.0044 | 3.2 | 4.0 | 10 | 30 | Neural tube development |
| GO:0001525 | 0.0046 | 1.9 | 14.6 | 25 | 109 | Angiogenesis |
| GO:0048856 | 0.0048 | 1.2 | 202.8 | 235 | 1,525 | Anatomical structure development |
| GO:0048646 | 0.0049 | 2.2 | 8.8 | 17 | 66 | Anatomical structure formation |
| GO:0000122 | 0.0055 | 1.7 | 21.1 | 33 | 157 | Negative regulation of transcription from RNA |
| | | | | | | polymerase II promoter |
| GO:0045595 | 0.0055 | 1.8 | 16.4 | 27 | 123 | Regulation of cell differentiation |
| GO:0007507 | 0.0063 | 1.8 | 16.5 | 27 | 123 | Heart development |
| GO:0000070 | 0.0065 | 4.1 | 2.4 | 7 | 18 | Mitotic sister chromatid segregation |
| GO:0021545 | 0.0067 | 4.8 | 1.8 | 6 | 14 | Cranial nerve development |
| GO:0006366 | 0.0070 | 1.3 | 59.7 | 78 | 448 | Transcription from RNA polymerase II promoter |
| GO:0048869 | 0.0073 | 1.2 | 149.1 | 176 | 1,107 | Cellular developmental process |
| GO:0008284 | 0.0076 | 1.5 | 28.1 | 41 | 209 | Positive regulation of cell proliferation |
| GO:0001708 | 0.0079 | 3.4 | 3.0 | 8 | 23 | Cell fate specification |
| GO:0007020 | 0.0081 | 8.5 | 0.9 | 4 | 7 | Microtubule nucleation |
| GO:0001655 | 0.0083 | 2.2 | 7.8 | 15 | 58 | Urogenital system development |
| GO:0001666 | 0.0083 | 2.2 | 7.8 | 15 | 58 | Response to hypoxia |
| GO:0000281 | 0.0087 | 19.3 | 0.5 | 3 | 4 | Cytokinesis after mitosis |
| GO:0009058 | 0.0088 | 1.1 | 405.0 | 442 | 3,007 | Biosynthetic process |
| GO:0035270 | 0.0093 | 2.5 | 5.7 | 12 | 43 | Endocrine system development |
| GO:0001649 | 0.0094 | 2.6 | 5.1 | 11 | 38 | Osteoblast differentiation |
| GO:0048699 | 0.0096 | 1.4 | 40.4 | 55 | 300 | Generation of neurons |
| GO:0007215 | 0.0099 | 4.2 | 2.0 | 6 | 15 | Glutamate signaling pathway |

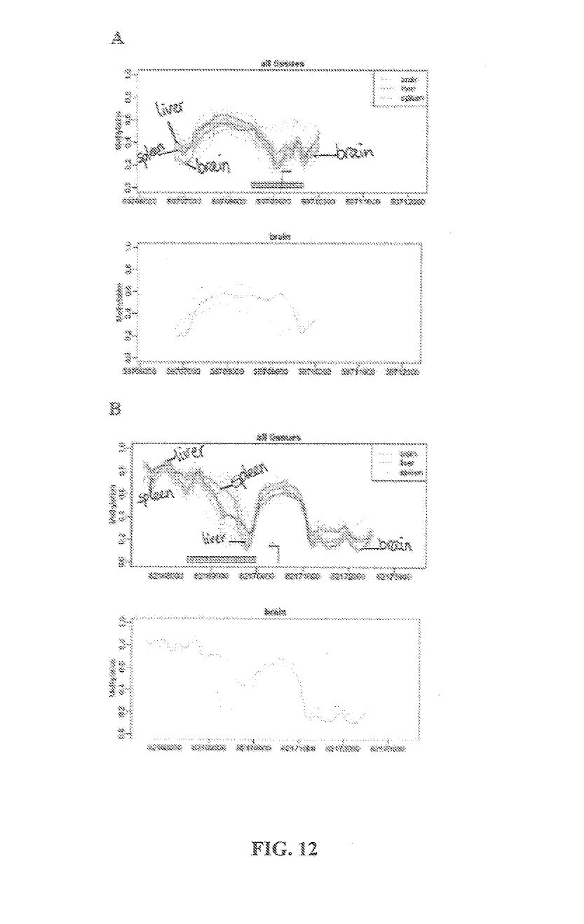

A similar analysis on mouse brain was performed. The results were even more striking. For example, FIG. 12 shows examples of developmental genes with VMRs in brains from isogenic mice raised in the same environment. Two examples of VMRs: Bmpr2, the receptor for the morphogenetic BMP protein, and Irs1, a key mediator of insulin-driven differentiation. Labeling is as in FIG. 10. The invention provides that VMRs are present across tissues and species, are enriched in development-related genes, and are related to phenotype, at least at the level of expression of the proximate gene.

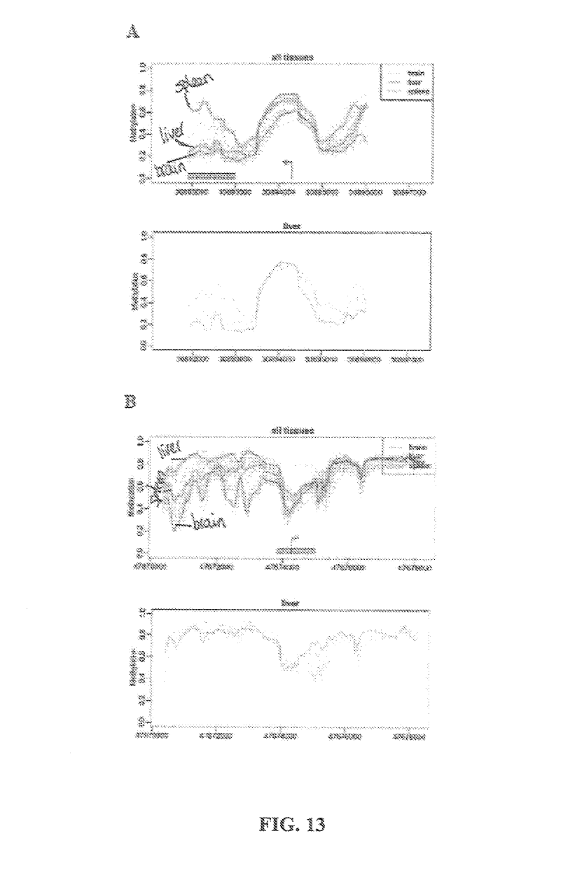

Also note that VMRs often are located near tissue-varying DMRs (T-DMRs), suggesting a mechanism by which they might evolve into each other over time. This is illustrated in FIG. 13 for mouse Ptp4a1, a protein tyrosine phosphatase involved in maintaining differentiated epithelial tissues, and for human FOXD2, a forkhead transcription factor involved in embryogenesis. Labeling is as in FIG. 10. In FIG. 13A, the VMR and T-DMR coincide, whereas in FIG. 13B, they are adjacent.

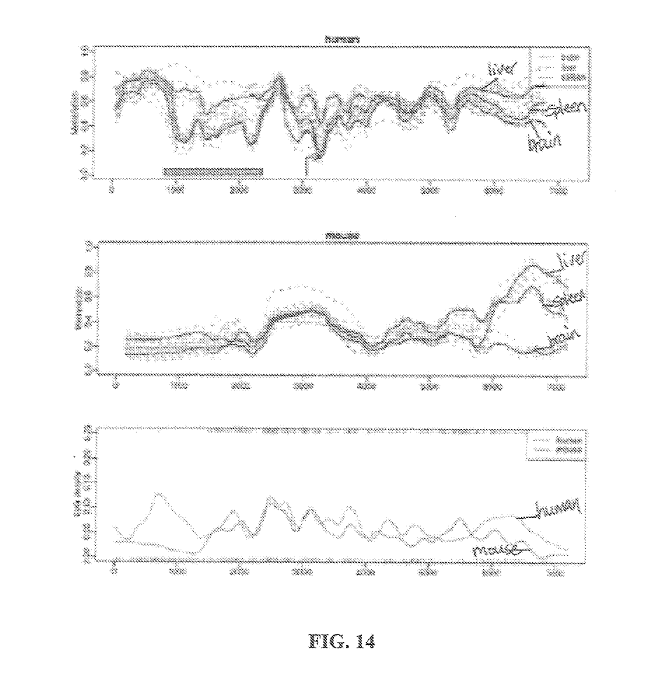

To address whether changes in differential methylation across species (mouse and human) can be traced back to an underlying genetic basis, the inventors focused on T-DMRs, given the wealth of data gathered in previous studies and their relevance to human diseases, such as cancer. DMRs are reported that distinguish colorectal cancer from normal colonic mucosa (C-DMRs) are enriched for T-DMRs, and this finding was validated in a large independent set of samples. In many cases, the loss of differential methylation in one species was related to an underlying loss of CpGs at the corresponding CpG island or nearby CpG island shore. A typical example of an evolutionary change in differential methylation involved LHX1, a transcriptional regulator essential for vertebrate head organization and mesoderm organization, (shown in FIG. 14). Note the T-DMR in human that is not in mouse on the left of the TSS. The human has gained CpGs at a CpG island shore (with the island shown in tick marks in the bottom panel). In contrast, both species have a moderate CpG count to the right of the TSS, and both have DMRs in this region. This is an example of how a genetic variation (i.e., gain of CpGs) allows for development-relevant tissue-specific differences in a highly conserved gene. Thus, differential methylation that itself differs across species may be due to underlying sequence variation at the site of these DMRs. Additional examples of this are available at rafalab.jhsph.edu/evometh.pdf.

FIG. 14 shows an underlying genetic basis for species differences in DMRs. A 7,500-bp human region was mapped to the mouse genome. The x-axis shows an index so that mapped bases are on top of one another. (Top) Methylation profiles for each human sample. As in FIG. 10, the dashed lines represent the individuals, and the solid lines represent the tissue averages. (Middle) The same plot for mouse. (Bottom) Ticks representing CpG locations for human and mouse. The ticks represent CpGs that were conserved. The curves represent CpG counts in a moving window of size 200 bases. Note that the lack of CpGs in the mouse at the beginning of the regions is associated with a difference in methylation patterns between species. Shown is LHX1, a transcriptional regulator essential for vertebrate head organization and mesoderm organization. Note the DMR in human that is not in mouse on the left of the TSS. The human has gained CpGs at a CpG island shore (tick marks). In contrast, both species have a moderate CpG count to the right of the TSS, and both have DMRs in this region.

Increased Stochastic Variation Would Increase Fitness in a Varying Environment. To model the role of epigenetic variation in natural selection, three simulations were performed based on a single quantitative phenotype that contributes to fitness, arbitrarily called Y. The inventors assumed that mutations of eight genomic locations affected the expected value of Y, with four mutations increasing Y and four decreasing Y. For two of the simulations (simulations 1 and 2), the inventors include a novel stochastic element controlled by eight mutations, four of which increased the variance of Y across the population given an identical genotype and four of which decreased this variance.

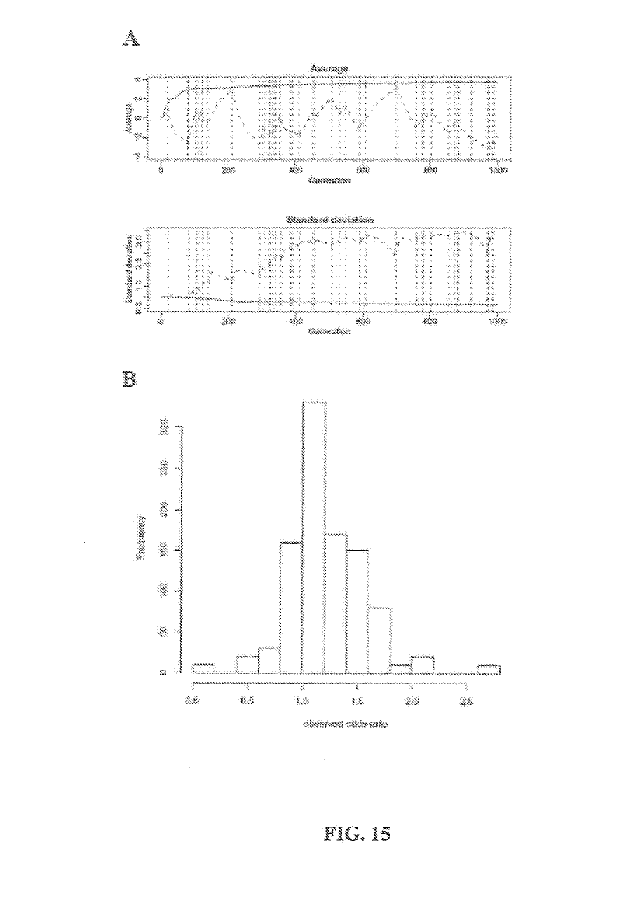

In simulation 1, the inventors emulated natural selection in a fixed environment favoring positive Y but including a novel stochastic epigenetic element, such that eight mutations affect the average of Y and eight mutations affect the variance of Y. As expected, this simulation favored the genotype with the largest expected value and the smallest variance (FIG. 15A). Simulation 2 is the same as simulation 1, but in this case the inventors allow a changing environment across generations that favor at times large Y and at times small Y. In this simulation, the most highly variable genotype is selected for and dominated by the 1,000th generation (FIG. 15A). In simulation 3, the inventors did not permit the variance to change. In this case, 72% of the iterations resulted in extinction before the 1,000th generation. This occurred because the genotype selected in one environment was not fit for the environment change after a dramatic environmental change. In contrast, when variance is allowed to change (simulation 2), extinction never occurred.

In addition, the inventors also emulated genome-wide association studies (GWAS) for Y. The individuals that do not survive were considered diseased, and the survivors are considered controls. An interesting finding is that the odds ratios for association between the genes known to affect fitness with disease hovered around 1.10 (FIG. 15B). The reason for this is because many of the diseased individuals are unfit only because of the affect of SNPs on variation, not because of the usual SNP-defined genetic change that directly affects function. This is simply a result of the low heritability that results from a large variance. Thus, the results of the epigenetic variation model are in agreement with results from current GWAS studies that explain very little attributable risk of disease.

FIG. 15 shows results of simulations demonstrating that increased stochastic variation in the epigenome would increase fitness in a varying environment. As discussed above, FIG. 15A depicts simulations of natural selection. For each simulation, the population average and SD of the phenotype are computed as a function of generation. Two simulations are shown: simulation 1, natural selection in a fixed environment favoring positive Y but including a novel stochastic epigenetic element, such that eight mutations affect average Y and eight mutations affect variance of Y, and simulation 2, similar to simulation 1 but in this case allowing a changing environment across generations that favor at times positive Y and at times negative Y. The top panel shows the average (across all iterations) population average of Y as a function of generation for simulation 1 (solid lines) and simulation 2 (dot lines). The dashed vertical lines indicate the generations at which the environment is changed in simulation 2. The bottom panel shows the average (across all iterations) population standard deviation of Y. Note that with a changing environment, the average Y fluctuates around a common point, but the SD of Y increases consistently. As discussed above, FIG. 15B is an emulation of GWAS analysis based on simulation 2 (varying variance of Y). Observed odds ratios are for SNPs that change the mean phenotype.

The methods and models provided herein propose that increased variability with a given genotype might increase fitness not by changing mean phenotype, but rather by changing the variability of phenotype with a given genotype. Also provided are possible mechanisms by which such enhanced variability can be genetically inherited and lead to increased stochastic epigenetic variation during development. Note that the genomic loci for such variation would be well defined in the model of the invention; examples of these loci are also provided. Although these loci do not represent the primary engine of development, they do provide plasticity in the developmental program by virtue of the stochastic variation that they impart through the genes in their proximity.

This methodology of the invention differs from that of a transgenerational epigenetic effect on phenotypic variation and disease risk described in Nadeau ((2009) Hum Mol Genet 18(R2):R202-210), in that in this model of the invention, the genetic variant is inherited and contributes to enhanced phenotypic variation, which can be mediated epigenetically in each generation. It also differs from a hypermutable genetic-switching model described in Salathe et al. ((2009) Genetics 182:1159-64)), in which the genotype itself changes from generation to generation, increasing phenotypic plasticity.

This methodology of the invention provides a mechanism for developmental plasticity and evolutionary adaptation to a fluctuating environment. Although the model is general and does not necessitate epigenetic variation, the invention provides the existence of VMRs that affect phenotype (i.e., gene expression) in isogenic mice raised in an identical environment, and have shown that similar VMRs exist in humans as well. A potential genetic mechanism is provided for differences in tissue-specific methylation across species—namely, the gain or loss of a CpG island or the associated shore. The localization near a specific gene can provide specificity of the effect of variation, but the mechanism for variation could entail the relationship to tissue-specific promoters, transcription factor binding sites, population variation in CpG density in these regions, or a combination of such factors. Distinguishing among these possibilities will require further experimentation.

Nonetheless, this methodology of the invention makes possible a specific prediction: that heritable genetic variation affects stochastic phenotypic variation. Thus, one should be able to identify SNPs that contribute to variance but not mean phenotype. Such SNPs do not necessitate an epigenetic mechanism for their influence, but at least some of them would be predicted to be in linkage disequilibrium to VMRs, such as those described above. The VMRs provide a possible mechanism for phenotypic variation in a given genetic background, and the inventors have direct evidence for this at least at the level of expression of the proximate gene. Some have also proposed that in a given environment, phenotypes eventually become genetically assimilated, and that the sequence differences in CpG islands and shores could provide a mechanism for both gain and loss in evolution of developmental variation mediated by DNA methylation.

This methodology of the invention and data provided differ from Lamarckianism, which argues that the environment modifies the genome. While not disputing the existence of such inheritance, the invention provides a genetic mechanism that may underlie this ability to vary epigenetically. The invention also departs from the neo-Darwinian and classical population genetics principle that heritable quantitative phenotypic variation is due entirely to the additive effect of individual trait loci. Here the heritable component is in part be a propensity to variation itself, adding an element of randomness to the phenotypic outcome. Thus, selection would be determined in part by the ability to vary around a setpoint, rather than by the setpoint itself. This notion is consistent with the idea of “order for free” described previously. Although the creators of that concept did not anticipate a role for epigenetics in evolution, inherent epigenetic variation itself will create new possibilities for ordered function—a question that now might be addressable mathematically, given the identification of a possible measurable substrate for this variation, namely DNA methylation. Of course, it remains unclear how much variation can be tolerated; at some point of increased variation, the individual species “identity” might deteriorate.

This methodology of the invention also may help explain observations in the evolutionary and epigenetic literature that have seemed paradoxical. In epigenetics, the apparent high degree of instability in the fidelity of epigenetic marks is puzzling. For example, cell lines propagated clonally are known to show a high frequency of random mono allelic expression. This epigenetic instability may have been first described while observing individual cancer cells, and data show clear epigenetic differences between identical twins. In evolutionary biology, social insects show environment-mediated phenotypic differences in social castes, and the distribution of those differences can be selected for, leading those authors to speculate that an epigenetic mechanism might be involved; the bee would be an outstanding model for testing these ideas. Further, substantial variations in phenotype of crayfish from an identical genotype have been reported. The authors also observed variable global DNA methylation, but as a phenotype, not a mechanism, and found no relationship between methylation and phenotype; they did not examine individual genes. The mechanism for phenotypic variation is epigenetic, and that increased variation would promote fitness.

Furthermore, not only variable phenotypes in normal tissue, but also variable disease phenotypes, might be obtained through inherent epigenetic variation. This is because a genetic variant providing a higher variance in phenotype also will increase the tails at both ends of the phenotype; that is, the same variant increasing fitness in one environment will increase the risk of decreasing fitness in a different environment. In support of this idea, DMRs are analyzed that are present in human but not in mouse, and many of these genes are found associated with human disorders of development as well as common complex diseases, including TAL1 (leukemia), FOXD3 (several disorders), HHEX (diabetes), PLCE1 (nephrotic syndrome), NKX2 (heart trunk malformation), TLX1 (leukemia), FEZ1 (esophageal cancer), ALX4 (forebrain absence), SHANK3 (brain/immune defect), NKX2 (heart malformations), and IGF2 (colorectal and other cancers). The inventors also note that in cancer the high degree of epigenetic variation (the mechanism of which has proved elusive) would follow directly from the evolutionary model of the invention. Thus, rather than arising from a varying environment acting across generations, cancer may arise in part from a repeatedly changing microenvironment due to, for example, repeated exposures to carcinogens, which would select for epigenetic heterogeneity, and thus the ability of cells to grow outside of their normal milieu.

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE 1

### Genetic Models

The mean model for the relationship between a quantitative phenotype and the genotype for a single locus is

E[pᵢ]=b₀+b_{AA}1(gᵢ=AA)+b_{Aa}1(gᵢ=AB)+b_{Aa}1(gᵢ=BB)+eᵢ

where pᵢ is the phenotype for individual i, gᵢ is the genotype, b₀ is the baseline level of the phenotype, 1(gᵢ=AA) is an indicator that the genotype for individual i is AA, b_{AA} is the phenotypic offset for allele AA and e is the random effect of other genetic, epigenetic, or environmental variables. The model relates the expected value (mean) of the phenotype to the genotype through a regression model (Fisher (1918) Trans R Soc Edinburgh 52:388-433). The model can be modified to specify additive and dominance effects, and to include the effect of multiple loci. This model is the basis for most common tests for association between genotype and phenotype (Walsh (1998) “Genetics and Nanalusis of Quantitative Traits,” Sunderland: Sinauer Associates). A mean SNP (mSNP) is a SNP where any of the b are nonzero.

The new model has the form:

Var[pᵢ]=c₀+c_{AA}1(gᵢ=AA)+c_{Aa}1(gᵢ=AB)+c_{Aa}1(gᵢ=BB)+εᵢ

where the variance of the phenotype is related to the genotype. In this model, c₀ is the baseline variance for the phenotype, c_{AA} is the change in variance due to the genotype AA, and 0ᵢ is the additional variability due to other genetic, environmental, or epigenetic variability. A variability SNP (vSNP) is a SNP where any of the c are nonzero.

### EXAMPLE 2

### Genetic Variability Test

To identify vSNPs, a studentized general regression based test was adapted for differences in variances using an unrestricted model (Breusch and Pagan (1979) Econometrica 47:1287-94). The first step in the statistical test is to fit the Fisher model by least squares and form the residuals

rᵢ=pᵢ−\hat{b}₀−\hat{b}₁1(gᵢ=AA)−\hat{b}₂1(gᵢ=AB)−\hat{b}₃1(gᵢ=BB)

with estimated residual variance

$${\hat{σ}}^{2} = \frac{1}{N} {\sum }_{i} {r}_{i}^{2} .$$

The standardized, squared residuals, ûᵢ=rᵢ²−\hat{σ}⁻² are regressed on the genotypes using the model

ûᵢ=c₀+c_{AA}1(gᵢ=AA)+c_{Aa}1(gᵢ=AB)+c_{Aa}1(gᵢ=BB) (1)

The test statistic is equal to nR² where n is the sample size and R² is the coefficient of determination for model (Fisher (1918) Trans R Soc Edinburgh 52:388-433). The test statistic is compared to the X²(k) distribution where k is one less than the number of unique genotypes.

### EXAMPLE 3

### Data Collection, Processing, and Adjustment for Surrogate Variables

Data Collection: Genotypes are obtained for 1,225 unrelated individuals with HBA1C measurements from the Genetics of Kidneys in Diabetes study. Patient recruitment and genotyping were performed as previously described (Mueller et al. (2006) J Am Soc Nephrol 17:1782-90). The dataset used for the analyses described in this manuscript are obtained from the database of Genotype and Phenotype (dbGaP) found on the world wide web at ncbi.nlm.nih.gov/gap through dbGaP accession number phs000018.v1.p1. Samples and associated phenotype data for the Search for Susceptibility Genes for Diabetic Nephropathy in Type 1 diabetes are provided by the Genetics of Kidneys in Diabetes Study, J. H. Warram of the Joslin Diabetes Center, Boston, Mass., USA (PI). Genotype data are obtained on the 210 unrelated HapMap individuals (hapmap.ncbi.nlm.nih.gov). Normalized genome-wide gene expression data are obtained on the same individuals from the Gene Expression Variation project (GENEVAR) (Stranger et al. (2005) PLoS Genet 1:e78). Sixty-four samples with high quality genome-scale DNA methylation data were taken from participants of the AGES Reykjavik Study.

Preprocessing: the inventors identified 1,225 unrelated individuals with measured hemoglobin A1C. The inventors analyzed only SNPs genotyped with a QC score greater than 0.99. The inventors also removed SNPs with a minor allele frequency less than 1% or with fewer than two unique genotypes, or where the least represented genotype represented fewer than 20 of the samples. Hemoglobin A1C measurements for the GoKind study are based on the Diabetes Control and Complications Trial standard and were not transformed. The inventors analyzed genotype data for the HapMap sample only for SNPs with at least two unique genotypes and with at least 10 samples per genotype. Gene expression data are collected, preprocessed, and normalized as previously described (Stranger et al. (2005) PLoS Genet 1:e78).

Adjustment for Surrogate Variables: Surrogate variables are estimates of latent confounders in gene expression data (Leek and Storey (2007) PloS Genet 3:1724-35). The inventors estimate surrogate variables in the HapMap gene expression data using the right singular values of the expression matrix. The adjusted analysis regresses the quantitative phenotype on both the genotypes and the surrogate variable estimates:

$${r}_{i}^{*} = {p}_{i} - {\hat{b}}_{0} - {\hat{b}}_{1} 1 ( {g}_{i} = AA ) - {\hat{b}}_{2} 1 ( {g}_{i} = AB ) - {\hat{b}}_{3} 1 ( {g}_{i} = BB ) - {\sum }_{j = 1}^{{n}_{sv}} {\hat{γ}}_{j} {\hat{s}}_{ji}$$

where ŝⱼᵢ is the estimated value for surrogate variable j for sample i. The next steps proceed as with the standard variability test; the residual variance is used to calculate the standardized squared residuals, which are regressed only on the genotypes:

û*ᵢ=d₀+d_{AA}1(gᵢ=AA)+d_{Aa}1(gᵢ=AB)+d_{Aa}1(gᵢ=BB)

The test statistic is equal to nR*² and is still compared to the x²(k) distribution where k is one less than the number of unique genotypes. There are 24 significant surrogate variables that are included in the analysis.

### EXAMPLE 4

### Data Analysis

GoKind: All SNPs that pass the preprocessing step are tested for association with hemoglobin A1C using both ANOVA and the variability test. The correlation between variability test p values and minor allele frequency is 0.01, suggesting the preprocessing filters are sufficient to remove any potential bias due to vary rare variants. The Benjamini-Hochberg algorithm is used to identify features significant at each false discovery rate threshold (Benjamini and Hochberg (1995) J of the Royal Statistical Society Series B—Methodological 57:289-300).

HapMap: All SNPs that pass the preprocessing steps are tested for association against the expression of the nearest gene using both ANOVA and the variability test. This approach treats each genes' expression as a quantitative trait. The ANOVA test is used to identify expression quantitative trait loci (eQTL), which have been extensively studied in both humans and other organisms (Schadt et al. (2003) Nature 422:297-302; Brem and Kruglyak (2005) PNAS USA 102:1572-77; Cheung et al. (2005) Nature 437:1365-69). The variability test identified SNPs that are associated with significant changes in the variability of gene expression, which are designated expression variable trait loci (eVTL).

The inventors categorize the SNPs into five groups based on their relationship to the nearest gene in terms of genomic distance. The five groups are: upstream (greater than 1000 bp away), in the promoter (within 1000 bp of transcription start), in an exon, in an intron, or downstream. The inventors also identify SNPs that are within 2000 bp of a CpG island or shore. For each of these categories, the inventors plot a histogram of the eVTL p-values within that category. Next the inventors pool the p-values into two groups (exon, promoter, CpG island/shore) and (intron, upstream, downstream). For each group the inventors calculate the proportion of P-values less than 0.05, then the inventors compute a test for differences in proportions.

Probe Mapping: Affymetrix annotation information is used to map SNPs to the nearest genes using cisGenome (Judy and Ji (2009) Bioinformatics 25:2369-75). Illumina probe locations are identified using the lumi R package (Du et al. (2008) Bioinformatics 24:1547-48).

### EXAMPLE 5

### Genotyping

5 ng of genomic DNA from primary non-immortalized lymphocytes is used for all genotyping assays. Pre-designed SNP assays from Applied Biosystems (Foster City, Calif.) are performed according to the manufacturer's recommendations, using GTXpress master mix on an ABI 7900 HT real-time PCR machine. The inventors examined FGF3, KCNQ1 and PER1 using assays C_{—}12040860_{—}10, C_{—}2278334_{—}10, and C_{—}9276979_{—}10, respectively, chosen for high heterozygosity and linkage disequilibrium in the CEPH dataset with both the vSNP identified in the GoKinD dataset and the VMRs in the tested sample set. Genotyping is determined using the ABI software.

Genome-wide screen for methylated human CpG islands has been disclosed, for example, in Strichman-Almashanu et al. (2002) Genome Research 12:543-54; the content of which is incorporated by reference in its entirety. For quantitative traits, the standard model for SNP association allows each genotype to have a different average value of the trait (Fisher (1918) Trans R Soc Edinburgh 52:399-433), to which the inventors refer here as mean-SNPs (mSNPs). This model is the basis for nearly every modern statistical test for genetic association including ANOVA, logistic regression, and interval mapping (Walsh (1008) “Genetics and Analysis of Quantitative Traits,” Sunderland: Sinauer Associates).

The model of the invention provides that variants exist commonly in which each genotype has a different variance, called variance-SNPs (vSNPs). This idea is fundamentally different from the usual concept of “genetic variability,” which refers to variability in the average values of the trait due to different alleles (Walsh (1008) “Genetics and Analysis of Quantitative Traits,” Sunderland: Sinauer Associates). For the vSNPs provided, a given allele is associated with a specific variability rather than with mean levels. This follows from the epigenetic model of the invention of stochastic variation, in which heritable variants control the degree of variation. This is fundamentally different than other important mechanisms for human disease, including rare variants (Dickson et al. (2010) PloS Biology 8:e1000294), copy number variation (McCarroll and Altshuler (2007) Nat Genet 39:S37-42), gene-gene interactions, and gene-environment interactions (Hunter (2005) Nat Rev Genet 6:287-98), where variability in the phenotype is explained by a complex combination of mean shifts attributable to interactions of measured genetic or environmental variables.

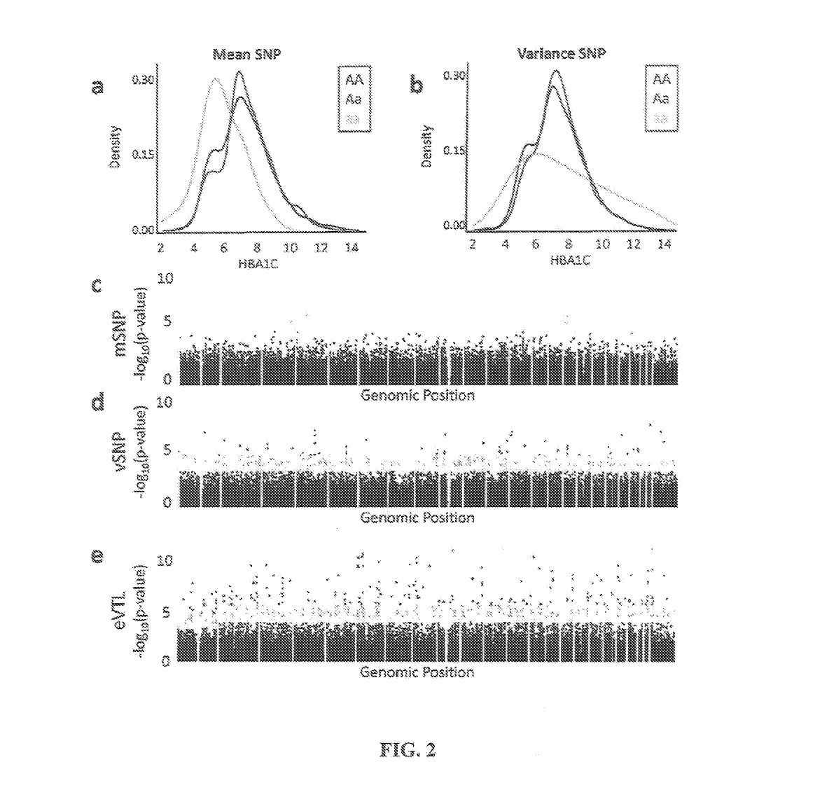

The inventors first tested for associations between mean levels of glycosylated hemoglobin (HbA1c) and genetic variation at 306,827 SNPs genotyped on 1,225 individuals in the GoKinD study (Mueller et al. (2006) J Am Soc Nephrol 17:1782-90), as is done in standard quantitative trait analyses (Walsh (1008) “Genetics and Analysis of Quantitative Traits,” Sunderland: Sinauer Associates). HbA1c is a measure of average plasma glucose concentration and is one of the benchmark measures for defining type I diabetes (Larsen et al. (1990) N Engl J. Med 323:1021-25). The inventors use a linear model to identify conventional mSNPs that are associated with a significant mean change in HbA1c. The linear model identifies 0, 5, and 12 mSNPs significant at false discovery rate thresholds of 1%, 5%, and 10% (example in FIG. 2A; all mSNPs in FIG. 2C).

As discussed above, FIG. 2 shows variability SNPs existing for HbA1c and gene expression traits. FIG. 2A is an example of a significant mean-SNP (mSNP) identified by analysis of the GoKinD dataset. The average HBA1C level is lower for individuals who received two copies of the minor allele, but the variance is unchanged.

FIG. 2C (mSNPs) and FIG. 2D (vSNPs): A plot of the −log₁₀ p-values versus genomic position (chromosomes 1-22, X ordered from left to right). For the mSNPs, 12, 5, and 0 are significant at a false discovery rate of 10%, 5%, and 1%, respectively. For the vSNPs, 607, 282, and 64 are significant at the same false discovery rates.

The inventors also test for associations between HbA1c variability (independent of mean) and genetic variation at the same SNPs; that vSNPs are searched in the same data. In genetics, there is no standard test for differences in variances between genotypes. The inventors therefore adapt the Breusch-Pagan test for differences in variance developed in econometrics. The variability test identifies 64, 282, and 607 significant vSNPs at the same false discovery rate thresholds (example in FIG. 2B; all vSNPs in FIG. 2D). Furthermore, 244 of the vSNPs significant at a 5% FDR have a minor allele frequency above 10%, suggesting that vSNPs for HbA1c are common variants.

To examine the functional significance of these vSNPs, gene ontology (GO) analysis is performed (Falcon and Gentleman (2007) Bioinformatics 23:257-58). Each SNP is associated with its closest genes in cisGenome (Judy and Ji (2009) Bioinformatics 25:2369-75). SNPs in gene deserts are removed from the analysis. For each GO category a hypergeometric test is performed to determine enrichment in the HbA1c vSNPs. This analysis results in 17 statistically significant categories that included pancreas development (p=0.002), regulation of glycoprotein biosynthetic process (p=0.002), regulation of polysaccharide metabolic process (p=0.007), proteoglycan metabolism (p=0.0004) and thymus development (p=0.01). These results are remarkably relevant to the pathophysiology of diabetes.

The second element of the stochastic epigenetic model of the invention provides that vSNPs affect the expression of proximate genes. It has already been conclusively shown that many associations exist between SNPs and the mean level of gene expression (Schadt et al. (2003) Nature 422:297-302; Brem and Kruglyak (2005) PNAS USA 102:1572-77); these associations have been referred to as expression quantitative trait loci (eQTL). Among eQTL, cis-eQTL are those that occur between a SNP and a proximate gene, and have been shown to have downstream functional effects (Emilsson et al. (2008) Nature 452:423-28). The inventors test for associations between the expression of 26,091 genes and 219,394 SNPs on the 210 unrelated HapMap individuals. The inventors treat the expression measurements for each of the 26,091 genes as a separate quantitative trait. The inventors test each SNP for association with variable expression of the gene whose coding region is closest to that SNP, resulting in the identification of 554 loci that the inventors refer to as expression variable trait loci (eVTL), corresponding to 273 unique genes at a false discovery rate of 5% (FIG. 2E).

As discussed above, FIG. 2 shows variability SNPs existing for HbA1c and gene expression traits. FIG. 2A is an example of a significant mean-SNP (mSNP) identified by analysis of the GoKinD dataset. The average HBA1C level is lower for individuals who received two copies of the minor allele, but the variance is unchanged. FIG. 2B is an example of a significant variance SNP (vSNP) by analysis of the GoKinD dataset. HbA1c levels are more variable for people who received two copies of the minor allele, α. FIG. 2C (mSNPs) and FIG. 2D (vSNPs): A plot of the −log₁₀ p-values versus genomic position (chromosomes 1-22, X ordered from left to right). For the mSNPs, 12, 5, and 0 are significant at a false discovery rate of 10%, 5%, and 1%, respectively. For the vSNPs, 607, 282, and 64 are significant at the same false discovery rates. FIG. 2E: The −log₁₀ p-values versus genomic position for expression variable trait loci (eVTL). Each SNP was mapped to the nearest gene and tested for association with variability of expression of that gene. There are 847, 554, and 235 eVTL significant at a false discovery rate of 10%, 5%, and 1%, respectively.

The inventors also assign each SNP to one of five categories according to their relationship to the nearest gene (upstream, promoter, exon, intron, and downstream), as well as within 1 kilobase of CpG islands/shores (Irizarry et al. (2009) Nat Genet 41:178-86). The eVTLs are most enriched near functional elements: exons, promoters, and CpG islands/shores, as compared to eVTLs in introns or upstream and downstream (P=4.84×10⁻¹¹). A GO analysis is also performed, as described above, that resulted in 123 categories. Interestingly, 42 of these categories are related to development or morphogenesis and 31 to development. These results are highly consistent with the GO annotation of stochastic epigenetic variation observed earlier.

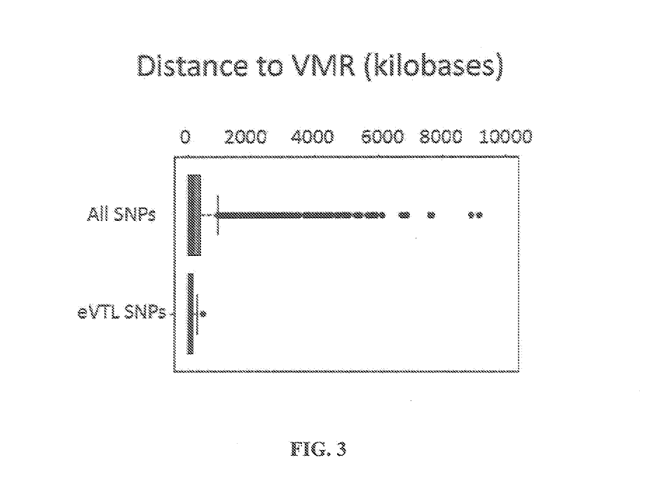

The third prediction of the model of the invention is that vSNPs will be in linkage disequilibrium with genomic locations harboring variably methylated regions (VMRs). In the model of the invention, these VMRs are functional elements that are selected for through evolution. To study the relationship between inherited variability and epigenetic variability, a genome-wide DNA methylation dataset derived from primary non-immortalized lymphocyte samples from 64 individuals is performed from the Age, Gene/Environment Susceptibility (AGES)-Reykjavik Study reported earlier (Bjornsson et al. (2008) JAMA 299:2877-83). Using the methods of the invention and criteria for VMR detection described earlier, the inventors identified within that dataset 2,500 VMRs. As predicted, eVTL SNPs identified in the HapMap individuals are significantly closer to VMRs than SNPs not associated with expression variability in this dataset, (FIG. 3), supporting the idea that vSNPs are in linkage disequilibrium with VMRs, and that they are common in the population.

FIG. 3 shows expression variable trait loci being located near variability methylated regions. Relationship of eVTL and VMRs: the top boxplot is the distribution of distances from all SNPs to VMRs, the bottom boxplot is the distribution of distances from eVTL to VMRs. eVTL are much closer to VMRs than are randomly selected SNPs.

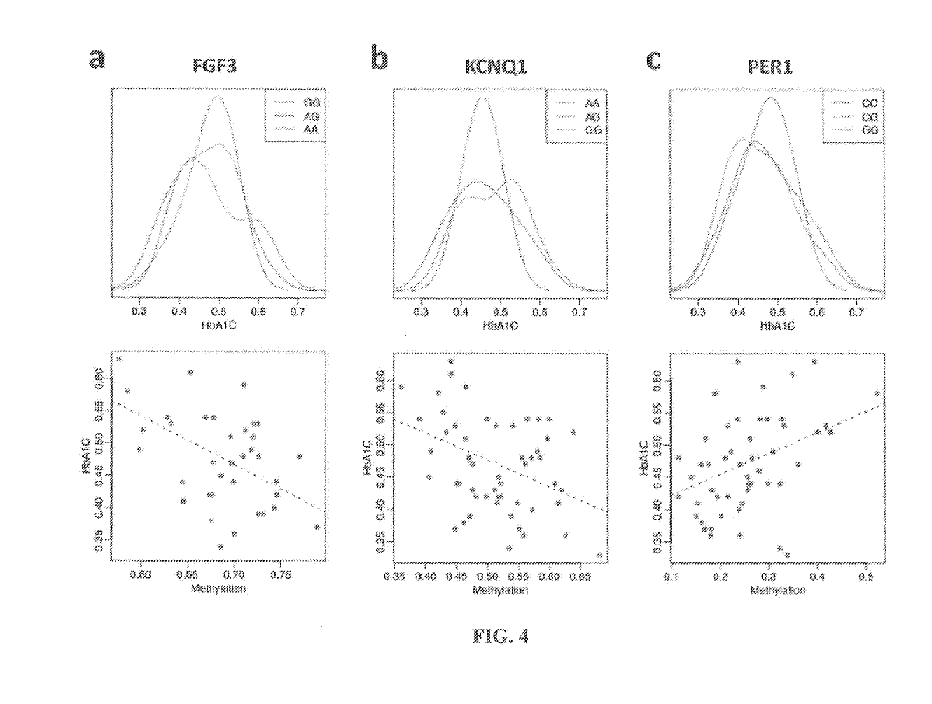

To confirm a direct relationship between genotype, variability in methylation, and variability in HBA1C, the inventors attempted to replicate the vSNP results in the sample set from which methylation data were available. The inventors identify 3 SNPs with high heterozygosity in this sample, lying within 10-78 kb and within the same linkage disequilibrium (LD) blocks as vSNPs identified using the GoKinD data, and also in the same LD blocks as VMRs that correlated with HbA1c. These SNPs are linked to genes implicated in diabetes, FGF3 (Todd (1997) Pathol Biol (Paris) 45:219-27), KCNQ1 (Qi et al. (2009) Hum Mol Genet 18:3508-15), and PER1 (Young et al. (2002) J Mol Cell Cardiol 34:223-231). The inventors also test whether these SNPs are vSNPs for HbA1c in this independent sample. For all 3 SNPs, the variance of HbA1c is genotype-dependent, but the mean levels are the same (FIG. 4, top panels), consistent with their being vSNPs. Furthermore, one can see that the relationship between HbA1c and DNA methylation is independent of genotype (FIG. 4, bottom panels). Applying the adapted Breusch-Pagan test to these data, two of the three vSNPs show a statistically significant dominance effect (P=0.02, 0.04, 0.14, corresponding FIG. 4A, B, C, respectively). Thus, vSNPs for HbA1c are in linkage disequilibrium with genomic locations harboring VMRs correlated with HbA1c.

FIG. 4 shows three HbA1c vSNPs showing variability effects in an independent sample of 65 individuals. The distribution of HbA1c (top panel) and relationship between HbA1c and methylation at VMRs in linkage disequilibrium (bottom panel) for three HbA1c vSNPs near genes FIG. 4A: FGF3; FIG. 4B: KCNQ1; and FIG. 4C: PER1. In all three cases, a copy of the minor allele leads to increased variability in HbA1c, but the relationship between HbA1c and methylation is consistent across genotypes.

### EXAMPLE 6

### Genome-Wide Methylation Assay

Samples: Non-immortalized lymphocyte samples are taken from participants of the AGES Reykjavik Study, which is described in detail elsewhere (Harris et al. (2007) Am J. Epidermiol 165:1076-87). 74 samples contribute to these analyses. These samples meet the high quality array data criteria and are from a randomly chosen set of 100 samples from the 638 AGES participants that have ample DNA from two visits. CHARM data are only considered in analyses if they pass the internal quality assessment of the invention. For cross-sectional analyses of the most recent collection (visit 7), 64 samples contribute data, while 48 contribute to cross-sectional analyses of the earlier visit 6 data. For identification of dynamic VMRs, a subset of 38 samples has quality CHARM data at both time points. For the analyses with BMI presented here, BMI is calculated as the body weight in kilograms (kg) divided by the height in meters (m) squared.

Genome-wide methylation assay: Comprehensive high-throughput array-based relative methylation (CHARM) analysis is performed, which is a microarray-based method agnostic to preconceptions about methylation, including location relative to genes and CpG content (Irizarry et al. (2008) Genome Res 18:780-90; Irizarry et al. (2009) Nat Genet 41:178-86). The resulting quantitative measurements of methylation, denoted with M, are log ratios of intensities from total (Cy3) and McrBC-fractionated DNA (Cy5): positive and negative M values are quantitatively associated with methylated and unmethylated sites, respectively. For each sample analyzed ˜4.5 million CpG sites across the genome using a custom designed NimbleGen HD2 microarray, including all of the classically defined CpG islands as well as non-repetitive progressively lower CpG density genomic regions of the genome until the array is saturated. The inventors include 4,500 control probes to standardize these M values so that unmethylated regions are associated, on average, with values of 0. CHARM is 100% specific at 90% sensitive for known methylation marks identified by other methods (e.g., in promoters), while including the more than half of the genome not identified by conventional region pre-selection. The CHARM results have also been extensively corroborated by quantitative bisulfite pyrosequencing analysis (Irizarry et al. (2008) Genome Res 18:780-90).

Identification of VMRs: The methylome for regions are screened where methylation varied substantially across individuals. The inventors term these variably methylated regions VMRs, to distinguish them from regions identified for their discrimination of groups, such as tissue types or cases versus controls, which are called DMRs. The use of the term VMR can be considered a specific type of metastable epi-allele introduced by Rakyan to denote variable expression of imprinted loci or variable methylation of an agouti methylation variant.

To identify VMRs from the data, the raw CHARM data are first processed with the statistical procedure described. This statistical procedure produced quality metrics (percent between 0-100) for each sample and, for those that pass the quality test of the invention (>80%), a vector of methylation percentage estimates for each feature on the array. These are then smoothed to reduce measurement error using the standard CHARM approach (Irizarry et al. (2009) Nat Genet 41:178-86). The inventors denote the resulting methylation percentages for subject i at microarray feature j for time t as Mᵢⱼₜ.

Cross-sectional analysis of visit 7 data is used to identify polymorphic variably methylated regions (VMRs) based on extreme inter-individual variance across consecutive probes. Specifically, the inventors estimate between subject variability using the median absolute deviation (MAD), a robust estimate of the standard deviation. The inventors computed the median of |Mᵢⱼₜ−mⱼₜ| across subjects, with mⱼₜ, the median Mᵢⱼₜ across subjects i, and referred to it as sⱼₜ. To avoid false positives in subsequent analysis of correlations with covariates, the inventors require a very stringent definition for designating a polymorphic VMR: a region of 10 or more consecutive probes attaining values of sⱼₜ above the 99^{th} percentile of all the sⱼₜ and an average sⱼₜ>0.125. The inventors chose these cut-off values using permutation tests. Specifically, the inventors randomize the genomic order of the CHARM probes and apply the above algorithm to find VMRs (including the smoothing step) for each permuted data set. Using the criteria of the invention, 0 false positives are obtained. Lowering either the number of consecutive probes or the average sⱼₜ thresholds can produce false positives. These VMRs are then annotated for genomic location and gene proximity. Genes within 3 kb of VMRs are considered in a GO analysis of biological process categories. For each GO category, a hypergeometric test is performed (Falcon and Gentleman (2007) Bioinformatrics 23:257-58), with corresponding nominal p value, to determine enrichment of genes near VMRs. The inventors also calculate the false discovery rate for each category statistic, to account for the multiple comparisons.

### EXAMPLE 7

### Identification of Stable Versus Dynamic VMRs

Methylation profiles for each sample are generated using the average Mᵢⱼₜ within the range of each VMR. This includes a vector of k VMR values for each subject i and time point t. The inventors calculate Dᵢₖ, the median absolute within-person difference between methylation profiles from visit 6 to visit 7 for each VMR k. A two component Gaussian mixture model is used to these values (Banfield and Raftery (1993) Biometrics 49:803-21) and use the resulting estimated posterior distributions to classify VMRs into three groups: “stable”: those with posterior probability of membership in the lower distribution>0.99, reflecting little intra-individual change over time; “dynamic”: those with posterior probability of membership in the higher distribution>0.99, reflecting those with high intra-individual change over time; and “ambiguous”: those not meeting either criteria, and thus in the overlap between the two distributions. (Note: Among the stable VMRs, there is some change over time observed in both directions, and when one takes the absolute value of this difference, the result is a small positive number, and thus the central tendency of Dₖ for stable VMRs is not zero.) To evaluate discrimination of individuals based on patterns, hierarchical clustering is applied to the vectors of methylation values for the VMRs and graphed individuals into a dendrogram based on similarity of VMRs. The inventors select only those VMRs designated as “stable” in the analysis above and repeated the hierarchical clustering and dendrogram graphic.

Identification of BMI-related methylated regions: Cross-sectional analyses for data at each visit is performed separately. For each stable VMR, a linear regression model is used to summarize the relationship between BMI and methylation. Specifically, for each VMR k, the inventors fit the following model:

Yᵢ=aₖ+bₖMᵢₖ+eᵢₖ

with Yᵢ is BMI for individual i, Mᵢₖ the methylation level for individual i in the k-th VMR, and e representing unexplained variability. Here bₖ represents the parameter of interest that summarizes the correlation between BMI and methylation. This produced one Wald-statistic for each VMR. The inventors fit this model to the data from visit 7 and to account for the multiple comparisons due to multiple VMRs, a list of regions with a false discovery rate of 0.30 is provided. To confirm these results, the inventors independently apply the same regression approach to visit 6 and obtained estimates of b along with p-values.

TABLE 4
Variably Methylated Regions Across Individuals

| | | | | | | | | Distance |
|---|---|---|---|---|---|---|---|---|
| | | | | | | | | from |
| | Start | End | Nearest | Visit | Visit | Change | Static vs | nearest |
| Chrom. | Position | Position | Gene | 7 SD | 6 SD | SD | Dynamic | gene |
| chrX | 39830089 | 39832051 | BCOR | 0.123 | 0.131 | 0.065 | static | 9548 |
| chrX | 39836616 | 39838366 | BCOR | 0.130 | 0.118 | 0.061 | static | 3233 |
| chrX | 72987615 | 72988745 | CHIC1 | 0.153 | 0.202 | 0.071 | static | 287847 |
| chrX | 39823122 | 39823821 | BCOR | 0.125 | 0.142 | 0.058 | static | 17778 |
| chr9 | 139164632 | 139165831 | GRIN1 | 0.127 | 0.137 | 0.064 | static | 11203 |
| chr3 | 22387326 | 22388136 | ZNF659 | 0.125 | 0.118 | 0.075 | static | 619507 |
| chr1 | 229178186 | 229178954 | TTC13 | 0.135 | 0.139 | 0.071 | static | 2252 |
| chrX | 39888311 | 39889238 | BCOR | 0.131 | 0.104 | 0.070 | static | 46712 |
| chrX | 139418948 | 139419933 | SOX3 | 0.133 | 0.113 | 0.068 | static | 4058 |
| chrX | 39846016 | 39846853 | BCOR | 0.144 | 0.133 | 0.049 | static | 4417 |
| chr17 | 73548137 | 73549033 | TNRC6C | 0.133 | 0.132 | 0.075 | static | 7555 |
| chrX | 39829126 | 39829738 | BCOR | 0.132 | 0.107 | 0.052 | static | 11861 |
| chr12 | 53004718 | 53005486 | COPZ1 | 0.129 | 0.098 | 0.140 | dynamic | 0 |
| chr20 | 3680979 | 3681951 | HSPA12B | 0.128 | 0.135 | 0.075 | static | 19624 |
| chrX | 39561298 | 39561889 | BCOR | 0.134 | 0.106 | 0.056 | static | 279710 |
| chr10 | 110214896 | 110215848 | SORCS1 | 0.126 | 0.118 | 0.067 | static | 1300615 |
| chr15 | 41879970 | 41880782 | HYPK | 0.127 | 0.082 | 0.110 | dynamic | 58 |
| chrX | 39835221 | 39835743 | BCOR | 0.142 | 0.143 | 0.059 | static | 5856 |
| chr7 | 112514377 | 112514899 | GPR85 | 0.131 | 0.105 | 0.084 | ambiguous | 115 |
| chrX | 39901201 | 39901818 | BCOR | 0.132 | 0.144 | 0.055 | static | 59602 |
| chr11 | 1861758 | 1862466 | LSP1 | 0.136 | 0.156 | 0.111 | dynamic | 13029 |
| chrX | 103696008 | 103696756 | IL1RAPL2 | 0.129 | 0.141 | 0.061 | static | 895 |
| chr7 | 129631923 | 129632661 | FLJ14803 | 0.122 | 0.135 | 0.101 | dynamic | 0 |
| chr1 | 204085619 | 204086111 | FLJ32569 | 0.183 | 0.220 | 0.074 | static | 0 |
| chr18 | 52964235 | 52964724 | WDR7 | 0.122 | 0.151 | 0.070 | static | 494622 |
| chr16 | 87080344 | 87081101 | ZFP1 | 0.142 | 0.103 | 0.053 | static | 32830 |
| chr6 | 167330879 | 167331377 | FGFR1OP | 0.126 | 0.107 | 0.058 | static | 1428 |
| chr1 | 148532753 | 148533318 | MRPS21 | 0.131 | 0.120 | 0.101 | dynamic | 0 |
| chr10 | 103040078 | 103040711 | LBX1 | 0.131 | 0.099 | 0.068 | static | 61372 |
| chr17 | 4646430 | 4646955 | PSMB6 | 0.148 | 0.138 | 0.116 | dynamic | 16 |
| chrX | 56807087 | 56807758 | UBQLN2 | 0.141 | 0.156 | 0.057 | static | 200291 |
| chr4 | 164471527 | 164472259 | NPY1R | 0.136 | 0.118 | 0.065 | static | 938 |
| chr10 | 134774107 | 134775511 | GPR123 | 0.133 | 0.107 | 0.072 | static | 39685 |
| chr6 | 26348556 | 26349220 | HIST1H4F | 0.125 | 0.099 | 0.112 | dynamic | 0 |
| chr7 | 27149232 | 27150278 | HOXA5 | 0.125 | 0.095 | 0.100 | dynamic | 0 |
| chrX | 13864694 | 13865346 | GPM6B | 0.134 | 0.136 | 0.080 | ambiguous | 1405 |
| chr14 | 23008449 | 23009184 | NGDN | 0.145 | 0.118 | 0.108 | dynamic | 0 |
| chr19 | 57181356 | 57181917 | ZNF350 | 0.119 | 0.093 | 0.109 | dynamic | 0 |
| chr20 | 19688587 | 19689214 | SLC24A3 | 0.121 | 0.130 | 0.100 | dynamic | 547298 |
| chr14 | 93323453 | 93324050 | PRIMA1 | 0.122 | 0.099 | 0.106 | dynamic | 468 |
| chr15 | 65143534 | 65144131 | SMAD3 | 0.146 | 0.112 | 0.081 | ambiguous | 1117 |
| chr10 | 99382542 | 99383137 | C10orf83 | 0.132 | 0.139 | 0.097 | ambiguous | 89 |
| chr19 | 57082309 | 57082870 | ZNF577 | 0.124 | 0.125 | 0.070 | static | 138 |
| chr8 | 81305424 | 81305985 | TPD52 | 0.130 | 0.096 | 0.109 | dynamic | 59034 |
| chr9 | 112841435 | 112841927 | EDG2 | 0.129 | 0.112 | 0.051 | static | 1250 |
| chr1 | 154170280 | 154170801 | KIAA0907 | 0.132 | 0.080 | 0.095 | ambiguous | 10 |
| chrX | 46964959 | 46965520 | PCTK1 | 0.131 | 0.112 | 0.114 | dynamic | 1901 |
| chr12 | 63850913 | 63851336 | LEMD3 | 0.143 | 0.155 | 0.065 | static | 1276 |
| chr1 | 145016136 | 145017015 | PRKAB2 | 0.132 | 0.121 | 0.068 | static | 93737 |
| chrX | 136484632 | 136485296 | ZIC3 | 0.122 | 0.139 | 0.066 | static | 8621 |
| chr1 | 19845043 | 19845628 | NBL1 | 0.120 | 0.130 | 0.081 | ambiguous | 1649 |
| chr4 | 122941359 | 122941779 | EXOSC9 | 0.161 | 0.167 | 0.061 | static | 142 |
| chrX | 69424498 | 69425027 | PDZD11 | 0.131 | 0.128 | 0.092 | ambiguous | 1569 |
| chr10 | 44679020 | 44679680 | RASSF4 | 0.125 | 0.124 | 0.054 | static | 95544 |
| chrY | 21975823 | 21977374 | RBMY1A1 | 0.138 | 0.120 | 0.043 | static | 105262 |
| chrX | 138602389 | 138602950 | ATP11C | 0.122 | 0.131 | 0.061 | static | 139162 |
| chr12 | 83830623 | 83831310 | SLC6A15 | 0.130 | 0.099 | 0.095 | ambiguous | 0 |
| chrX | 24929339 | 24929831 | ARX | 0.133 | 0.127 | 0.053 | static | 13943 |
| chr6 | 139054547 | 139054967 | CCDC28A | 0.136 | 0.166 | 0.051 | static | 81382 |
| chr11 | 9289755 | 9290247 | TMEM41B | 0.144 | 0.123 | 0.054 | static | 2624 |
| chr17 | 52477866 | 52478658 | SCPEP1 | 0.141 | 0.117 | 0.112 | dynamic | 67379 |
| chr4 | 184255275 | 184255767 | FLJ30277 | 0.133 | 0.091 | 0.061 | static | 1578 |
| chr22 | 17660061 | 17660586 | CLTCL1 | 0.155 | 0.094 | 0.058 | static | 823 |
| chr18 | 18183689 | 18184211 | CTAGE1 | 0.123 | 0.102 | 0.060 | static | 67664 |
| chr12 | 52430514 | 52431174 | CALCOCO1 | 0.127 | 0.107 | 0.063 | static | 23029 |
| chrX | 85291108 | 85291685 | DACH2 | 0.130 | 0.141 | 0.060 | static | 828 |
| chrX | 48928427 | 48929350 | LMO6 | 0.133 | 0.090 | 0.063 | static | 369 |
| chr5 | 43073696 | 43074185 | LOC389289 | 0.126 | 0.128 | 0.085 | ambiguous | 1912 |
| chr3 | 46992745 | 46993273 | CCDC12 | 0.141 | 0.156 | 0.115 | dynamic | 0 |
| chrX | 56809402 | 56810136 | UBQLN2 | 0.135 | 0.132 | 0.090 | ambiguous | 202606 |
| chr5 | 95321436 | 95321921 | ELL2 | 0.128 | 0.120 | 0.069 | static | 1609 |
| chrX | 74059895 | 74060467 | KIAA2022 | 0.124 | 0.139 | 0.043 | static | 1241 |
| chr2 | 45251338 | 45251840 | SIX2 | 0.122 | 0.107 | 0.074 | static | 161313 |
| chr11 | 58697996 | 58698500 | FAM111A | 0.132 | 0.103 | 0.068 | static | 29169 |
| chrX | 39832159 | 39832699 | BCOR | 0.133 | 0.139 | 0.066 | static | 8900 |
| chr12 | 51975276 | 51975798 | PFDN5 | 0.142 | 0.145 | 0.100 | dynamic | 0 |
| chr19 | 42156067 | 42156592 | ZNF568 | 0.122 | 0.099 | 0.077 | ambiguous | 56994 |
| chr22 | 18115899 | 18116316 | TBX1 | 0.139 | 0.124 | 0.078 | ambiguous | 7909 |
| chr16 | 74159074 | 74159611 | GABARAPL2 | 0.120 | 0.133 | 0.087 | ambiguous | 1325 |
| chr2 | 216655441 | 216655930 | TMEM169 | 0.123 | 0.108 | 0.059 | static | 555 |
| chr10 | 52505394 | 52505850 | PRKG1 | 0.133 | 0.108 | 0.062 | static | 1096 |
| chr3 | 35656017 | 35656532 | ARPP-21 | 0.134 | 0.079 | 0.122 | dynamic | 2320 |
| chr5 | 1157734 | 1158499 | SLC12A7 | 0.133 | 0.129 | 0.060 | static | 6609 |
| chr4 | 90446622 | 90447102 | GPRIN3 | 0.124 | 0.094 | 0.089 | ambiguous | 1081 |
| chr5 | 83053906 | 83054362 | HAPLN1 | 0.128 | 0.108 | 0.135 | dynamic | 1453 |
| chr19 | 4921792 | 4922437 | JMJD2B | 0.137 | 0.123 | 0.065 | static | 1661 |
| chr17 | 697008 | 697530 | NXN | 0.122 | 0.090 | 0.065 | static | 132229 |
| chr19 | 45006529 | 45007123 | DYRK1B | 0.124 | 0.129 | 0.089 | ambiguous | 9557 |
| chrX | 37963673 | 37964138 | SRPX | 0.136 | 0.100 | 0.071 | static | 936 |
| chr15 | 91164324 | 91164961 | CHD2 | 0.124 | 0.101 | 0.111 | dynamic | 79461 |
| chrX | 133513150 | 133513647 | PLAC1 | 0.128 | 0.120 | 0.056 | static | 106531 |
| chr12 | 51370883 | 51371637 | KRT77 | 0.124 | 0.095 | 0.075 | ambiguous | 11876 |
| chr6 | 39388575 | 39389028 | KCNK17 | 0.133 | 0.086 | 0.070 | static | 1185 |
| chr6 | 29725667 | 29726054 | MOG | 0.121 | 0.090 | 0.090 | ambiguous | 6733 |
| chr8 | 19657619 | 19657934 | INTS10 | 0.160 | 0.152 | 0.073 | static | 61263 |
| chrX | 149904173 | 149904632 | HMGB3 | 0.142 | 0.124 | 0.060 | static | 1753 |
| chr5 | 114543459 | 114544229 | TRIM36 | 0.122 | 0.083 | 0.071 | static | 0 |
| chr19 | 7661645 | 7662169 | FCER2 | 0.140 | 0.163 | 0.135 | dynamic | 10827 |
| chr19 | 54646572 | 54646992 | NOP17 | 0.123 | 0.097 | 0.072 | static | 0 |
| chr16 | 64076952 | 64077300 | CDH11 | 0.181 | 0.214 | 0.079 | ambiguous | 363533 |
| chr14 | 67157306 | 67157696 | ARG2 | 0.145 | 0.137 | 0.055 | static | 975 |
| chr19 | 50575060 | 50575480 | PPP1R13L | 0.132 | 0.171 | 0.073 | static | 24648 |
| chr9 | 135513445 | 135514006 | DBH | 0.152 | 0.122 | 0.061 | static | 22140 |
| chr13 | 113918445 | 113918757 | RASA3 | 0.142 | 0.112 | 0.057 | static | 2249 |
| chrX | 119326721 | 119327249 | FAM70A | 0.133 | 0.142 | 0.067 | static | 2066 |
| chr7 | 126677154 | 126677646 | GRM8 | 0.121 | 0.103 | 0.065 | static | 6609 |
| chr10 | 97658091 | 97658547 | ENTPD1 | 0.123 | 0.120 | 0.071 | static | 152186 |
| chr10 | 31934137 | 31934626 | TCF8 | 0.123 | 0.153 | 0.094 | ambiguous | 285990 |
| chr1 | 226700071 | 226700491 | HIST3H2A | 0.133 | 0.133 | 0.083 | ambiguous | 11691 |
| chrX | 103386571 | 103387094 | ESX1 | 0.128 | 0.090 | 0.072 | static | 328 |
| chr14 | 44502647 | 44503064 | KIAA0423 | 0.122 | 0.137 | 0.082 | ambiguous | 1482 |
| chr5 | 177986111 | 177986564 | CLK4 | 0.138 | 0.119 | 0.137 | dynamic | 95 |
| chr2 | 118660218 | 118660635 | INSIG2 | 0.139 | 0.126 | 0.122 | dynamic | 97699 |
| chr16 | 85651774 | 85652280 | FBXO31 | 0.125 | 0.141 | 0.063 | static | 322583 |
| chr12 | 109371874 | 109372291 | ARPC3 | 0.140 | 0.106 | 0.082 | ambiguous | 249 |
| chr6 | 30176316 | 30176775 | TRIM31 | 0.124 | 0.107 | 0.085 | ambiguous | 12070 |
| chrX | 8660712 | 8661221 | KAL1 | 0.129 | 0.106 | 0.078 | ambiguous | 486 |
| chr12 | 9986709 | 9987195 | FLJ46363 | 0.134 | 0.100 | 0.048 | static | 10007 |
| chr1 | 21597015 | 21597604 | NBPF3 | 0.125 | 0.124 | 0.082 | ambiguous | 41613 |
| chr17 | 44036829 | 44037352 | HOXB6 | 0.138 | 0.136 | 0.068 | static | 0 |
| chr21 | 33326315 | 33326807 | OLIG2 | 0.120 | 0.096 | 0.051 | static | 6207 |
| chrX | 103699727 | 103700150 | IL1RAPL2 | 0.123 | 0.096 | 0.068 | static | 2076 |
| chr8 | 114519845 | 114520324 | CSMD3 | 0.125 | 0.135 | 0.070 | static | 1428 |
| chr8 | 819442 | 819823 | ERICH1 | 0.121 | 0.136 | 0.071 | static | 148217 |
| chr20 | 43369626 | 43370213 | RBPSUHL | 0.121 | 0.120 | 0.053 | static | 722 |
| chr1 | 208130230 | 208130542 | C1orf107 | 0.134 | 0.117 | 0.087 | ambiguous | 62275 |
| chr14 | 85065117 | 85065471 | FLRT2 | 0.130 | 0.143 | 0.073 | static | 769 |
| chr7 | 100025680 | 100025998 | FBXO24 | 0.146 | 0.124 | 0.082 | ambiguous | 3789 |
| chr17 | 5613998 | 5614340 | NALP1 | 0.142 | 0.128 | 0.077 | ambiguous | 185446 |
| chrX | 49574659 | 49575007 | LOC158572 | 0.132 | 0.155 | 0.057 | static | 44201 |
| chr17 | 10039348 | 10039765 | GAS7 | 0.121 | 0.142 | 0.076 | ambiguous | 2827 |
| chr6 | 85539926 | 85540454 | KIAA1009 | 0.125 | 0.077 | 0.105 | dynamic | 545894 |
| chr1 | 231008549 | 231008903 | C1orf57 | 0.130 | 0.108 | 0.055 | static | 144089 |
| chr10 | 44680118 | 44680466 | RASSF4 | 0.154 | 0.130 | 0.081 | ambiguous | 94758 |
| chr11 | 62277871 | 62278361 | ZBTB3 | 0.125 | 0.125 | 0.084 | ambiguous | 0 |
| chr3 | 56809743 | 56810127 | ARHGEF3 | 0.124 | 0.114 | 0.079 | ambiguous | 865 |
| chr15 | 43195152 | 43195539 | DUOXA2 | 0.126 | 0.065 | 0.081 | ambiguous | 1337 |
| chr18 | 10445682 | 10446072 | APCDD1 | 0.129 | 0.127 | 0.056 | static | 1058 |
| chr9 | 85024122 | 85024473 | FRMD3 | 0.126 | 0.109 | 0.087 | ambiguous | 318694 |
| chr14 | 68326882 | 68327239 | ZFP36L1 | 0.126 | 0.142 | 0.108 | dynamic | 2298 |
| chrX | 39886872 | 39887220 | BCOR | 0.132 | 0.119 | 0.056 | static | 45273 |
| chr1 | 36387479 | 36387932 | TRAPPC3 | 0.129 | 0.108 | 0.121 | dynamic | 0 |
| chr10 | 51846766 | 51847221 | TMEM23 | 0.127 | 0.123 | 0.068 | static | 206521 |
| chr5 | 159368733 | 159369319 | TTC1 | 0.128 | 0.108 | 0.111 | dynamic | 0 |
| chr19 | 49022520 | 49023043 | LYPD5 | 0.123 | 0.120 | 0.074 | static | 5895 |
| chr17 | 84181 | 84667 | RPH3AL | 0.120 | 0.126 | 0.053 | static | 117908 |
| chr14 | 28304596 | 28305118 | FOXG1B | 0.128 | 0.116 | 0.127 | dynamic | 919 |
| chr8 | 587304 | 588116 | LOC389607 | 0.120 | 0.117 | 0.048 | static | 13502 |
| chr1 | 150348334 | 150349435 | TCHHL1 | 0.123 | 0.129 | 0.082 | ambiguous | 20171 |
| chr5 | 75732495 | 75732843 | IQGAP2 | 0.127 | 0.137 | 0.092 | ambiguous | 2061 |
| chr8 | 42475258 | 42475645 | SLC20A2 | 0.129 | 0.105 | 0.067 | static | 40579 |
| chr4 | 109306333 | 109306681 | LEF1 | 0.121 | 0.142 | 0.113 | dynamic | 2345 |
| chr7 | 104410767 | 104411212 | MLL5 | 0.125 | 0.100 | 0.080 | ambiguous | 30660 |
| chr12 | 116888436 | 116888935 | RFC5 | 0.126 | 0.131 | 0.050 | static | 49957 |
| chr2 | 176701658 | 176702114 | HOXD8 | 0.131 | 0.133 | 0.097 | ambiguous | 608 |
| chrX | 48978649 | 48978966 | CCDC22 | 0.129 | 0.147 | 0.084 | ambiguous | 0 |
| chr2 | 118486367 | 118486745 | CCDC93 | 0.124 | 0.099 | 0.081 | ambiguous | 1421 |
| chr17 | 24193098 | 24193585 | C17orf63 | 0.127 | 0.121 | 0.099 | ambiguous | 381 |
| chr10 | 69279262 | 69279578 | DNAJC12 | 0.126 | 0.129 | 0.099 | ambiguous | 11320 |
| chr8 | 118032803 | 118033249 | LOC441376 | 0.122 | 0.096 | 0.070 | static | 13140 |
| chrX | 72584559 | 72584943 | CDX4 | 0.129 | 0.117 | 0.055 | static | 745 |
| chr22 | 22643605 | 22643968 | DDT | 0.124 | 0.109 | 0.090 | ambiguous | 8050 |
| chr12 | 129217129 | 129217480 | FZD10 | 0.129 | 0.090 | 0.072 | static | 4145 |
| chr20 | 11615539 | 11615923 | BTBD3 | 0.131 | 0.128 | 0.112 | dynamic | 203553 |
| chr16 | 75784419 | 75784770 | MON1B | 0.123 | 0.110 | 0.080 | ambiguous | 2083 |
| chr7 | 31341305 | 31341653 | NEUROD6 | 0.127 | 0.138 | 0.070 | static | 5409 |
| chr12 | 52667120 | 52667468 | HOXC10 | 0.121 | 0.109 | 0.080 | ambiguous | 1908 |
| chr19 | 6483683 | 6484032 | TNFSF9 | 0.122 | 0.134 | 0.092 | ambiguous | 1647 |
| chrX | 50231114 | 50231596 | DGKK | 0.121 | 0.143 | 0.074 | static | 638 |
| chr3 | 139635024 | 139635492 | FAM62C | 0.127 | 0.126 | 0.070 | static | 616 |
| chr11 | 131446958 | 131447270 | HNT | 0.129 | 0.110 | 0.047 | static | 161037 |
| chr12 | 79628117 | 79628429 | MYF6 | 0.146 | 0.139 | 0.080 | ambiguous | 2541 |
| chr7 | 1876647 | 1877835 | MAD1L1 | 0.135 | 0.150 | 0.072 | static | 361273 |
| chrX | 104952198 | 104952690 | NRK | 0.122 | 0.126 | 0.087 | ambiguous | 501 |
| chr10 | 99200125 | 99200491 | ZDHHC16 | 0.121 | 0.105 | 0.095 | ambiguous | 4189 |
| chr1 | 58815736 | 58816340 | TACSTD2 | 0.122 | 0.151 | 0.073 | static | 0 |
| chr6 | 43128953 | 43129301 | CUL7 | 0.123 | 0.089 | 0.087 | ambiguous | 330 |
| chr8 | 67513737 | 67514159 | ADHFE1 | 0.119 | 0.116 | 0.086 | ambiguous | 6450 |
| chr18 | 8693428 | 8693840 | KIAA0802 | 0.124 | 0.090 | 0.054 | static | 13528 |
| chr22 | 49015075 | 49015531 | TUBGCP6 | 0.138 | 0.136 | 0.070 | static | 9995 |
| chr7 | 27100723 | 27101104 | HOXA1 | 0.141 | 0.120 | 0.055 | static | 1045 |
| chr3 | 54133707 | 54134058 | CACNA2D3 | 0.123 | 0.107 | 0.075 | static | 1975 |
| chr17 | 45941279 | 45941591 | MYCBPAP | 0.123 | 0.103 | 0.061 | static | 469 |
| chr17 | 4745363 | 4745708 | CHRNE | 0.124 | 0.083 | 0.067 | static | 1439 |
| chr15 | 41572599 | 41573019 | TP53BP1 | 0.122 | 0.120 | 0.079 | ambiguous | 17008 |
| chrX | 119034082 | 119034628 | PEPP-2 | 0.122 | 0.116 | 0.079 | ambiguous | 61106 |
| chr6 | 26138282 | 26138666 | HIST1H3B | 0.125 | 0.117 | 0.111 | dynamic | 1600 |
| chrX | 20341021 | 20341336 | RPS6KA3 | 0.128 | 0.125 | 0.077 | ambiguous | 146351 |
| chrX | 48341363 | 48341741 | WDR13 | 0.124 | 0.116 | 0.070 | static | 519 |
| chr7 | 1165166 | 1165964 | ZFAND2A | 0.126 | 0.114 | 0.077 | ambiguous | 359 |
| chr16 | 7076946 | 7077297 | A2BP1 | 0.127 | 0.100 | 0.090 | ambiguous | 1067814 |
| chr18 | 65221152 | 65221470 | DOK6 | 0.119 | 0.105 | 0.054 | static | 1882 |
| chrX | 21304583 | 21304897 | CNKSR2 | 0.136 | 0.104 | 0.061 | static | 1683 |
| chrX | 72583680 | 72584067 | CDX4 | 0.127 | 0.116 | 0.049 | static | 0 |
| chr16 | 3170698 | 3171075 | OR1F1 | 0.133 | 0.148 | 0.063 | static | 23172 |
| chr11 | 4586227 | 4586628 | TRIM68 | 0.121 | 0.104 | 0.092 | ambiguous | 215 |
| chr2 | 79074019 | 79074373 | REG3G | 0.127 | 0.133 | 0.073 | static | 31960 |
| chr22 | 40416193 | 40416508 | FLJ23584 | 0.129 | 0.121 | 0.095 | ambiguous | 0 |
| chr6 | 72652573 | 72652891 | RIMS1 | 0.126 | 0.118 | 0.131 | dynamic | 479 |
| chr14 | 98780250 | 98780658 | BCL11B | 0.128 | 0.157 | 0.104 | dynamic | 26916 |
| chr6 | 41451610 | 41451922 | NCR2 | 0.130 | 0.084 | 0.069 | static | 40106 |
| chr8 | 26777997 | 26778309 | ADRA1A | 0.130 | 0.116 | 0.076 | ambiguous | 529 |
| chr6 | 11262380 | 11262791 | NEDD9 | 0.149 | 0.116 | 0.063 | static | 78092 |
| chr15 | 40660724 | 40661120 | CEP27 | 0.121 | 0.113 | 0.114 | dynamic | 32380 |
| chrX | 48866532 | 48866955 | GPKOW | 0.126 | 0.135 | 0.069 | static | 67 |
| chrX | 83330251 | 83330613 | RPS6KA6 | 0.124 | 0.107 | 0.066 | static | 3995 |
| chr19 | 3325403 | 3325757 | NFIC | 0.122 | 0.099 | 0.081 | ambiguous | 7831 |
| chrX | 135058281 | 135058595 | FHL1 | 0.124 | 0.110 | 0.067 | static | 936 |
| chr5 | 88007250 | 88007634 | MEF2C | 0.125 | 0.097 | 0.109 | dynamic | 207145 |
| chr15 | 38451885 | 38452200 | DISP2 | 0.130 | 0.142 | 0.105 | dynamic | 14160 |
| chr6 | 36499296 | 36499614 | KCTD20 | 0.129 | 0.115 | 0.097 | ambiguous | 18907 |
| chr13 | 77392256 | 77392571 | EDNRB | 0.125 | 0.136 | 0.066 | static | 55093 |
| chr7 | 129920089 | 129920404 | MEST | 0.133 | 0.104 | 0.076 | ambiguous | 916 |
| chr4 | 48181513 | 48181828 | SLC10A4 | 0.131 | 0.129 | 0.068 | static | 1308 |
| chr17 | 44036304 | 44036616 | HOXB6 | 0.126 | 0.115 | 0.082 | ambiguous | 716 |
| chr9 | 70927348 | 70927802 | FXN | 0.123 | 0.103 | 0.069 | static | 87185 |
| chr7 | 2705109 | 2705424 | AMZ1 | 0.122 | 0.108 | 0.083 | ambiguous | 19421 |
| chr15 | 21446448 | 21446763 | NDN | 0.128 | 0.130 | 0.103 | dynamic | 36779 |
| chr17 | 34860430 | 34860778 | PPARBP | 0.120 | 0.120 | 0.108 | dynamic | 251 |
| chr10 | 102017088 | 102017403 | CWF19L1 | 0.126 | 0.145 | 0.108 | dynamic | 23 |
| chr17 | 37967632 | 37967974 | COASY | 0.130 | 0.131 | 0.111 | dynamic | 15 |
| chrX | 40677549 | 40678047 | USP9X | 0.120 | 0.099 | 0.065 | static | 151784 |
| chr20 | 44073597 | 44073909 | MMP9 | 0.123 | 0.123 | 0.071 | static | 2644 |
| chr11 | 47693153 | 47693473 | AGBL2 | 0.122 | 0.086 | 0.102 | dynamic | 276 |
| chr19 | 59298233 | 59298548 | NDUFA3 | 0.125 | 0.079 | 0.096 | ambiguous | 262 |
| chr7 | 157893737 | 157894124 | PTPRN2 | 0.128 | 0.097 | 0.078 | ambiguous | 179054 |
| chr19 | 42649497 | 42649809 | ZNF569 | 0.123 | 0.117 | 0.077 | ambiguous | 369 |

### EXAMPLE 8

### Selection Model Simulations

Tissue Samples and CHARM: Human tissues are obtained from the Stanley Foundation, and mouse tissues from C57BL/6 wild-type mice were obtained from Jackson Laboratory. Sample preparation and the CHARM DNA methylation analysis from which the data sets are derived are described in more detail elsewhere (Irizarry et al. (2009) Nat Genet 41:178-86; Irizarry et al. (2008) Genome Res 18:780-90).

VMRs: First, the microarray raw data from CHARM arrays (Irizarry et al. (2009) Nat Genet 41:178-86) were transformed into estimated methylation percentages for each genomic location represented by a probe. These values were then smoothed (Irizarry et al. (2009) Nat Genet 41:178-86) to obtain estimated methylation profiles for each sample. Then for each tissue, the SD for each location is computed. A region of locations surpassing a 99.95% percentile of all of the variances is designated a VMR.

Simulations: To create the simulation, the inventors expanded the Fisher-Wright neutral selection model. In the neutral model, the inventors start with N individuals and to create the next generation, the inventors select N individuals at random with replacement. This implies that the number of children for each individual follows a multinomial distribution, with population size remaining fixed at N. To introduce selection, the inventors permitted each individual to die with probability 1−pₙ, with the survival probability pₙ depending on a phenotype, Yₙ. For the next generation, the inventors selected N individuals, with replacement, from those that survived. For the simulation shown here, the inventors quantified this relationship with a simple logistic function, log{pₙ 1(1−pₙ)}=a+bYₙ. Note that if b is positive, then positive Y individuals are more fit, and if b is negative, then negative Y individual are more fit. The inventors assumed the existence of M SNPs, Xₘ, m=1, . . . , M, that affect the phenotype. The inventors assumed two possible polymorphisms, designated 0 and 1, and denote the expected change on the phenotype by βⱼ, j=1, . . . , M. The inventors referred to (X₁, . . . , X_{M}) as the genotype. Note that there are 2^{M} different genotypes.

The inventors followed Fisher's additive model for complex traits and assumed that the phenotype was a random variable with

Yₙ=β₁X_{n,1}+β₂X_{n,2}+ . . . +β_{M}X_{n,M}+eₙ.

Here e represents variation not explained by the standard genetic model and assumed to be a Gaussian random quantity with mean 0 and standard deviation s. Note that each genotype will have a different average Y value, determined by the effects β. The inventors added an epigenetic variation term caused by sequence changes (e.g., the addition of a CpG island that allows the presence of a VMR or T-DMR). The inventors model this by incorporating another feature; the inventors assume the existence of M SNPs that altered the individual's variability (i.e., changed s). This is the epigenetic scenario, in which the inventors are incorporating sequence variation that affects the variability of the phenotype, without altering the mean of the phenotype. This would be analogous to the earlier examples of loss or gain of CpGs that lead to the loss or gain of differentially methylated regions. The inventors denote this epigenetic variation-inducing sequence change by Z and the effects by y, and assume

Log 2(Sₙ)=γ₁Z_{n,1}+γ₂Z_{n,2}+ . . . +γₘZ_{n,m}.

Simulation 1: The inventors started this simulation with an isogenic population and permit mutations to occur independently and at random at rate r. This simulation is ran with n=10,000, a=−4, b=4, M=8 with (β₁, . . . , β₈)=(−1, −1, −1, −1, 1, 1, 1), s=1, and r=10⁻⁴. Note that these values of a and b imply that a average individual (Y=0) has about a 1% chance of surviving. In contrast, an individual with the (0, 0, 0, 0, 1, 1, 1, 1) genotype has about a 99% chance of surviving. For the epigenetic part of the model of the invention, the inventors use (y₁, . . . , y₈)=(−1, −1, −1, −1, 1, 1, 1, 1)/2. This implies that some mutations increase phenotype variance by 50% and others decrease it by 50%. The inventors run 1,000 generations 250 times.

Simulation 2, environment changing: Simulation 1 is repeated except that dramatic environmental changes are used to change the environment and its relationship with phenotype and fitness. The occurrence of these events is assumed to be random at a rate of 1 per 25 generations. Such a change results in b changing from 4 to −4. This implies that after the first event, smaller-than-average individuals were more fit than taller-than-average individuals. To check whether the outcome was stable, the inventors considered a more skewed initial condition. Specifically, the original simulation is repeated using 12 different sets of initial parameters. The number of iterations is increased to 5,000. The inventors varied the environment changing rate to be 1 per 5, 1 per 10, 1 per 25, or 1 per 50 generations. Further, the number of mutating SNPs is varied to be 2, 8, or 16. The conclusions from these simulations are as expected: Variability increases fitness, particularly in a changing environment.

Simulation 3: Simulation 3 is the same as simulation 1, except the inventors did not permit mutations to affect the variance of Y.

Although the invention has been described with reference to the above examples, it will be understood that modifications and variations are encompassed within the spirit and scope of the invention. Accordingly, the invention is limited only by the following claims.

What is claimed is:

1. A method of predicting risk for a condition or disorder in a subject, comprising:
(a) measuring the expression level of at least one expression variable trait loci (eVTL) in a biological sample from the subject;
(b) measuring the methylation level of at least one variably methylated region (VMR) correlated with at least one variability genotype in a biological sample from the subject; and
(c) predicting the risk for the condition or disorder in the subject based on the expression level of the eVTL in (a) and the methylation level measured in (b).

2. The method of claim 1, further comprising the step of:
performing an association study between a genotype variability information and a gene expression variability information, thereby identifying at least one variability genotype associated with the selected gene expression.

3. The method of claim 2, further comprising the step of:
performing an association study between each of the at least one variability genotype and a genome-wide gene expression data, thereby identifying at least one expression variable trait loci (eVTL), wherein the at least one eVTL is associated with the condition or disorder.

4. The method of claim 1, wherein the condition or disorder is diabetes.

5. The method of claim 1, wherein the at least one variably methylated region (VMR) correlated with the variability genotype is selected from the group consisting of FGF3, KCNQ1 and PER1.

6. The method of claim 1, wherein the at least one variably methylated region (VMR) correlated with the variability genotype comprises FGF3, KCNQ1 and PER1.

7. A method of predicting risk for a condition or disorder in a subject, comprising:
(a) obtaining genotype data from a plurality of samples;
(b) obtaining genome-wide gene expression data from the samples;
(c) performing a first variability test for the genotype data, thereby obtaining genotype variability information;
(d) performing a second variability test for at least one selected gene expression from the samples, thereby obtaining gene expression variability information, wherein the selected gene expression correlates with the condition or disorder;
(e) performing a first association study between the genotype variability information of (c) and the gene expression variability information of (d), thereby identifying at least one variability genotype associated with the selected gene expression;
(f) performing a second association study between each of the at least one variability genotype identified in (e) and the genome-wide gene expression data of (b), thereby identifying at least one expression variable trait loci (eVTL), wherein the at least one eVTL is associated with the condition or disorder;
(g) identifying a plurality of variably methylated regions (VMRs) correlated with the selected gene expression;
(h) performing a linkage disequilibrium (LD) study between the at least one variability genotype identified in (e) and the VMRs correlated with the selected gene expression identified in (g), thereby identifying at least one VMR correlated with the variability genotype;
(i) measuring expression level of the at least one eVTL in (f) in a biological sample from the subject;
(j) measuring methylation level of the at least one VMR correlated with the variability genotype identified in (g) in a biological sample from the subject; and
(k) predicting the risk for the condition or disorder in the subject based on the expression level of the eVTL in (i) and the methylation level measured in (j).

8. The method of claim 7, further comprises a step of performing a third association study between the genotype data of (a) and the selected gene expression from the samples, thereby identifying at least one mean genotype associated with the selected gene expression.

9. The method of claim 8, wherein the at least one mean genotype associated with the gene expression comprises at least one mean SNP or mSNP.

10. The method of claim 7, further comprises a step of performing a gene ontology analysis for each of the at least one variability genotype.

11. The method of claim 10, wherein the gene ontology analysis is Gostats.

12. The method of claim 7, wherein the genotype data comprises single nucleotide polymorphism (SNP) data.

13. The method of claim 7, wherein the at least one selected gene expression comprises levels of hemoglobin HbA1c.

14. The method of claim 7, wherein the first or second variability test is Breusch-Pagan test.

15. The method of claim 7, wherein the at least one variability genotype associated with the gene expression comprises at least one variability SNP or vSNP.

16. The method of claim 7, wherein the variably methylated regions (VMRs) correlated with the selected gene expression is selected from the group consisting of FGF3, KCNQ1, and PER1.

17. The method of claim 7, wherein the variably methylated regions (VMRs) correlated with the selected gene expression comprise FGF3, KCNQ1, and PER1.

18. The method of claim 7, wherein the at least one variably methylated region (VMR) correlated with the variability genotype is selected from the group consisting of FGF3, KCNQ1, and PER1.

19. The method of claim 7, wherein the at least one variably methylated region (VMR) correlated with the variability genotype comprises FGF3, KCNQ1, and PER1.

20. A method for analyzing epigenetic information, using suitable computer software for use on a computer, comprising:
(a) performing a first variability test for genotype data obtained from a plurality of samples, thereby obtaining genotype variability information;
(b) performing a second variability test for at least one selected gene expression from the samples, thereby obtaining gene expression variability information;
(c) performing a first association study between the genotype variability information of (a) and the gene expression variability information of (b), thereby identifying at least one variability genotype associated with the selected gene expression;
(d) performing a second association study between each of the at least one variability genotype identified in (c) and genome-wide gene expression data obtained from the samples, thereby identifying at least one expression variable trait loci (eVTL); and
(e) performing a linkage disequilibrium (LD) study between the at least one variability genotype identified in (c) and a plurality of variably methylated regions (VMRs) correlated with the selected gene expression, thereby identifying at least one VMR correlated with the variability genotype.

21. The method of claim 20, further comprises the step of performing a third association study between the genotype data and the selected gene expression from the samples, thereby identifying at least one mean genotype associated with the selected gene expression.

22. The method of claim 20, further comprises a step of performing a gene ontology analysis for each of the at least one variability genotype.

23. A system for identifying expression variable trait loci (eVTL) and variably methylated regions (VMRs) for predicting risk for a condition or disorder in a subject, comprising:
(a) a first variability module performing a first variability test for genotype data obtained from a plurality of samples, thereby obtaining genotype variability information;
(b) a second variability module performing a second variability test for at least one selected gene expression, thereby obtaining gene expression variability information, wherein the selected gene expression correlates with the condition or disorder;
(c) a first association module performing a first association study between the genotype variability information of (a) and the gene expression variability information of (b), thereby identifying at least one variability genotype associated with the selected gene expression;
(d) a second association module performing a second association study between each of the at least one variability genotype identified in (c) and genome-wide gene expression data obtained from the samples, thereby identifying at least one expression variable trait loci (eVTL); and
(e) a linkage disequilibrium module performing a linkage disequilibrium (LD) study between the at least one variability genotype identified in (c) and a plurality of VMRs correlated with the selected gene expression, thereby identifying at least one VMR correlated with the variability genotype.

24. The system of claim 23, further comprises a third association module performing a third association study between the genotype data and at least one selected gene expression from the samples, thereby identifying at least one mean genotype associated with the selected gene expression, wherein the selected gene expression correlates with the condition or disorder.

25. The method of claim 23, further comprises a gene ontology module performing a gene ontology analysis for each of the at least one variability genotype.

26. A method for predicting risk for a condition or disorder in a subject, comprising:
(a) measuring intra-sample change over time for genome-wide variably methylated regions (VMRs) from a plurality of samples;
(b) performing gene ontology analysis for the VMRs;
(c) identifying at least one VMR correlated with the condition or disorder using a linear regression model;
(d) measuring methylation level of the at least one VMRs correlated with the condition or disorder in a biological sample from the subject; and
(e) predicting the risk for the condition or disorder in the subject based on the methylation level measured in (d).

27. The method of claim 26, wherein the condition or disorder is body mass index (BMI).

28. The method of claim 26, wherein the change over time is a change over 11 years.

29. The method of claim 26, wherein the at least one VMR correlated with the condition or disorder is selected from the group consisting of MMP9, PRKG1, RFC5, CACNA2D3, and PM20D1.

30. The method of claim 26, wherein the at least one VMR correlated with the condition or disorder comprises MMP9, PRKG1, RFC5, CACNA2D3, and PM20D1.

31. The method of claim 26, wherein the at least one VMR correlated with the condition or disorder has at least one nearest gene selected from the group consisting of IL1RAPL2, PM2OD1, NEDD9, MMP9, SORCS1, PRKG1, RFC5, TTC13, DACH2, TRIM36, FLRT2, C1orf57, and APCDD1.

32. The method of claim 26, wherein IL1RAPL2, PM2OD1, NEDD9, MMP9, SORCS1, PRKG1, RFC5, TTC13, DACH2, TRIM36, FLRT2, C1orf57, and APCDD1 are nearest genes to the at least one VMR correlated with the condition or disorder.

33. A method for generating an epigenetic signature for a subject, comprising:
(a) measuring intra-sample change over time for genome-wide variably methylated regions (VMRs) from a plurality of samples;
(b) separating selected VMRs into two groups using a two component Gaussian mixture model based on the measured intra-sample change of (a), wherein the VMRs in the higher distribution are designated as dynamic VMRs and the VMRs in the lower distribution are designated as stable VMRs;
(c) measuring methylation levels of a plurality of stable VMRs in a biological sample from the subject; and
(d) generating the epigenetic signature for the subject based on the methylation levels measured in (c).

34. The method of claim 33, wherein methylation levels of at least five stable VMRs of the subject are measured.

35. The method of claim 33, wherein the stable VMRs are selected from the group consisting of MMP9, PRKG1, RFC5, CACNA2D3, and PM20D1.

36. The method of claim 33, wherein the stable VMRs comprise MMP9, PRKG1, RFC5, CACNA2D3, and PM20D1.

37. The method of claim 33, wherein the stable VMRs have at least one nearest gene selected from the group consisting of IL1RAPL2, PM2OD1, NEDD9, MMP9, SORCS1, PRKG1, RFC5, TTC13, DACH2, TRIM36, FLRT2, C1orf57, and APCDD1.

38. The method of claim 33, wherein IL1RAPL2, PM2OD1, NEDD9, MMP9, SORCS1, PRKG1, RFC5, TTC13, DACH2, TRIM36, FLRT2, C1orf57, and APCDD1 are nearest genes to the stable VMRs.

39. A method for simulating epigenetic plasticity across generations, comprising:
(a) generating a plurality of genotype variants, wherein the genotype variants are genetically inherited;
(b) applying natural selection favoring a first subset of the genotype variants;
(c) enabling a plurality of stochastic epigenetic elements, wherein the stochastic epigenetic elements change phenotypes without changing the genotype variants;
(d) allowing a changing environment across generations favoring a second subset of the genotype variants; and
(e) monitoring fluctuations of mean phenotype across generations.

40. The method of claim 39, further comprising the step of:
comparing frequency of fitness from genome-wide association study (GWAS) with the genotype variants which change the mean phenotype.

41. The method of claim 39, wherein a Fisher-Wright neutral selection model is used.

42. The method of claim 39, wherein a Fisher's additive model is used.

43. The method of claim 39, wherein a multinomial distribution is used.

44. The method of claim 39, wherein each of the genotype variants has two possible polymorphisms.

45. The method of claim 39, wherein the stochastic epigenetic elements represent additions or deletions of CpG islands.

46. The method of claim 39, wherein the method uses suitable computer software for use on a computer.

47. A plurality of nucleic acid sequences, selected from the group consisting of variably methylated region (VMR) sequences as set forth in Table 4, and any combination thereof.

48. The plurality of nucleic acid sequences of claim 47, wherein the plurality is a microarray.

49. A kit for detecting risk of a condition or disorder comprising a plurality of oligonucleotide primer sequences capable of generating a plurality of amplificates from genomic DNA, the amplificates consisting of variably methylated region (VMR) sequences as set forth in Table 4, and any combination thereof.

50. The kit of claim 49, further comprising instructions for detecting risk.

51. The kit of claim 50, wherein the condition or disorder is diabetes or obesity.

52. The kit of claim 49, further comprising instructions for detecting risk and computer executable code for performing statistical analysis.
